(19) United States
(12) Patent Application Publication
(10) Pub. No.: US 2019/0076409 A1
(43) Pub. Date: Mar. 14, 2019

(54) USE OF GABOXADOL IN THE TREATMENT OF NARCOLEPSY

(71) Applicant: Ovid Therapeutics Inc., New York, NY (US)

(72) Inventor: Matthew DURING, Weston, CT (US)

(21) Appl. No.: 16/127,375

(22) Filed: Sep. 11, 2018

Related U.S. Application Data
(60) Provisional application No. 62/557,412, filed on Sep. 12, 2017.

(51) Int. Cl. A61K 31/437; A61P 25/00

(52) U.S. Cl. CPC A61K 31/437; A61K 31/137; A61K 2300/00; A61P 25/00

(57) ABSTRACT

Methods and compositions for treating narcolepsy are provided which include administering gaboxadol or a pharmaceutically acceptable salt thereof to a patient diagnosed with narcolepsy. Also provided are methods and compositions for treating narcolepsy which include administering to a patient in need thereof gaboxadol or a pharmaceutically acceptable salt thereof, in combination with one or more of a CNS stimulant, a eugeroic agent, an antidepressant or a GABA_{B} receptor agonist.

### CROSS REFERENCE TO RELATED APPLICATIONS

This application claims benefit of and priority to U.S. Provisional Application No. 62/557,412, filed Sep. 12, 2017, which is incorporated herein by reference in its entirety.

### BACKGROUND

### 1. Technical Field

Treatment of narcolepsy with gaboxadol is provided.

### 2. Description of Related Art

Narcolepsy is a chronic neurological disorder involving a decreased ability to regulate sleep-wake cycles. The most typical symptoms are excessive daytime sleepiness, abnormal REM sleep, cataplexy, sleep paralysis, and hallucinations. Other symptoms may include automatic behaviors and night-time wakefulness. Not all symptoms appear in all patients.

In a normal sleep cycle, a person enters rapid eye movement (REM) sleep after about 60 to 90 minutes. Dreams occur during REM sleep, and the brain keeps muscles limp during this sleep stage. People with narcolepsy frequently enter REM sleep rapidly, within 15 minutes of falling asleep. Also, the muscle weakness or dream activity of REM sleep can occur during wakefulness or be absent during sleep.

In narcolepsy, excessive daytime sleepiness (EDS) may last from seconds to minutes or longer and can occur at any time. EDS is characterized by persistent sleepiness, regardless of how much sleep a person gets at night. Sleepiness in narcolepsy can have a sudden onset in that an overwhelming sense of sleepiness comes on quickly. In between sleep attacks, individuals may have normal levels of alertness.

A majority of those affected with narcolepsy also experience episodes of sudden loss of muscle strength, known as cataplexy. Cataplexy is often triggered by sudden, strong emotions such as laughter, fear, anger, stress, or excitement. Cataplexy may appear weeks or even years after the onset of EDS. In certain cases, patients may only have one or two attacks in a lifetime, while others may experience many attacks a day. Attacks may be mild and involve only a momentary sense of minor weakness in a limited number of muscles, such as a slight drooping of the eyelids. The most severe attacks can result in a total body collapse during which individuals are unable to move, speak, or keep their eyes open. However, even during the most severe episodes, people remain fully conscious, a characteristic that distinguishes cataplexy from fainting or seizure disorders. In some instances, speech can be slurred and vision may be impaired (double vision, inability to focus), while hearing and awareness remain normal. The loss of muscle tone during cataplexy may resemble paralysis of muscle activity that naturally occurs during REM sleep. Cataleptic episodes usually last just a few minutes and resolve quickly on their own.

Sleep paralysis associated with narcolepsy is the temporary inability to talk or move when waking or when falling asleep. It usually lasts for a few seconds to minutes. Sleep paralysis resembles cataplexy except it occurs at the edges of sleep. As with cataplexy, people remain conscious.

Hallucinations associated with narcolepsy can be quite vivid and frightening images can accompany sleep paralysis. Such hallucinations usually occur when people are falling asleep or waking up. Most often the content is primarily visual or auditory, but any of the other senses can be involved.

Automatic behaviors associated with narcolepsy occur when a person continues to function (talking, putting things away, etc.) during temporary sleep episodes but awakens with no memory of performing such activities. For example, a person may fall asleep during an activity (e.g., eating, talking) and automatically continues the activity for a few seconds or minutes without conscious awareness of what they are doing. This happens most often while people are engaged in habitual activities such as typing or driving.

While individuals with narcolepsy are very sleepy during the day, they usually also experience difficulties staying asleep at night, i.e., fragmented sleep. Sleep may be disrupted by insomnia, vivid dreaming, sleep apnea, acting out while dreaming, and periodic leg movements.

Narcolepsy has been categorized into two major types. Type 1 narcolepsy was previously termed narcolepsy with cataplexy. This diagnosis is based on the individual either having low levels of hypocretin, a brain hormone, or reporting cataplexy and having excessive daytime sleepiness on a special nap test. Hypocretin promotes wakefulness and regulates REM sleep. Type 2 narcolepsy was previously termed narcolepsy without cataplexy. People with this condition experience excessive daytime sleepiness but usually do not have muscle weakness triggered by emotions. They usually also have less severe symptoms and normal levels of hypocretin.

Although there is no cure for narcolepsy, symptoms are treatable with medications and lifestyle adjustments. Central nervous system (CNS) stimulants are frequently used to treat ESD. For example, methylphenidate, amphetamines such as methamphetamine and dextroamphetamine, and eugeroics such as modafinil and armodafiinil. However, side effects associated with CNS stimulants can include anxiety, headaches, loss of appetite and tolerance. Antidepressant medications may also be used, e.g., to control cataplexy. Two classes of antidepressant drugs used to treat narcoleptic cataplexy are tricyclic antidepressants which include imipramine, desipramine, clomipramine, and protriptyline, and selective serotonin and noradrenergic reuptake inhibitors which include venlafaxine, fluoxetine, and atomoxetine. In general, antidepressants produce fewer adverse effects than CNS stimulants. However, troublesome side effects can still occur in some individuals, including impotence, high blood pressure, and heart rhythm irregularities. Another treatment option for narcolepsy is sodium oxybate, also known as sodium gamma-hydroxybutyrate (GHB), a GABA_{B} receptor agonist. It can be used for cataplexy associated with narcolepsy and excessive daytime sleepiness associated with narcolepsy. Sodium oxybate is a strong sedative that is typically taken at night. Due to safety concerns associated with the use of this drug, the distribution of sodium oxybate is tightly restricted. There remains a need for effective treatments for narcolepsy.

Gaboxadol (4,5,6,7-tetrahydroisoxazolo [5,4-c] pyridine-3-ol) (THIP)) is described in U.S. Pat. Nos. 4,278,676, 4,362,731, 4,353,910, and WO 2005/094820 is a selective GABA_{A} receptor agonist with a preference for δ-subunit containing GABA_{A} receptors. In the early 1980s gaboxadol was the subject of a series of pilot studies that tested its efficacy as an analgesic and anxiolytic, as well as a treatment for tardive dyskinesia, Huntington's disease, Alzheimer's disease, and spasticity. In the 1990s gaboxadol moved into late stage development for the treatment of insomnia but failed to show significant effects in sleep onset and sleep maintenance in a three-month efficacy study. Additionally, patients with a history of drug abuse who received gaboxadol experienced a steep increase in psychiatric adverse events. As a result of these negative results the development of gaboxadol was terminated.

### SUMMARY

A method of treating narcolepsy is provided which includes administering to a patient in need thereof gaboxadol or a pharmaceutically acceptable salt thereof. Also provided is a method of treating narcolepsy which includes administering to a patient in need thereof gaboxadol or a pharmaceutically acceptable salt thereof, in combination with one or more of a CNS stimulant, a eugeroic agent, an antidepressant or a GABA_{B} receptor agonist.

Methods of treating narcolepsy described herein also include administering to a patient in need thereof gaboxadol or a pharmaceutically acceptable salt thereof wherein the method provides improvement in one or more symptoms of narcolepsy. Methods of treating narcolepsy described herein also include administering to a patient in need thereof gaboxadol or a pharmaceutically acceptable salt thereof wherein the method provides improvement in next day functioning of the patient. Methods of treating narcolepsy described herein also include administering to a patient in need thereof gaboxadol or a pharmaceutically acceptable salt thereof in an amount effective to delay onset of REM sleep in the patient after falling asleep. Methods of treating narcolepsy described herein also include administering to a patient in need thereof gaboxadol or a pharmaceutically acceptable salt thereof wherein the method provides improvement in the patient for more than 4, 6, 8, 10, 12, 14, 16, 18, 20, 22, or 24 or more hours after administration to the patient.

Methods of treating narcolepsy described herein also include administering to a patient in need thereof gaboxadol or a pharmaceutically acceptable salt thereof in combination with one or more of a CNS stimulant, a eugeroic agent, an antidepressant or a GABA_{B} receptor agonist, wherein the method provides improvement in one or more symptoms of narcolepsy. Methods of treating narcolepsy described herein also include administering to a patient in need thereof gaboxadol or a pharmaceutically acceptable salt thereof in combination with one or more of a CNS stimulant, a eugeroic agent, an antidepressant or a GABA_{B} receptor agonist, wherein the method provides improvement in next day functioning of the patient. Methods of treating narcolepsy described herein also include administering to a patient in need thereof gaboxadol or a pharmaceutically acceptable salt thereof in combination with one or more of a CNS stimulant, a eugeroic agent, an antidepressant or a GABA_{B} receptor agonist, wherein the method provides improvement in the patient for more than 4, 6, 8, 10, 12, 14, 16, 18, 20, 22, 24 or more hours after administration to the patient.

### BRIEF DESCRIPTION OF THE DRAWING

FIG. 1 shows the arithmetic mean plasma concentration-time profiles of gaboxadol following single oral doses (2.5, 5, 10, 15, and 20 mg) as described in Example 1 with horizontal lines Δ indicating the change between 6 and 12 hours.

### DETAILED DESCRIPTION

Described herein are methods of treating narcolepsy with gaboxadol or a pharmaceutically acceptable salt thereof. Methods of treating narcolepsy described herein include administering to a patient in need thereof gaboxadol or a pharmaceutically acceptable salt thereof wherein the method provides improvement in one or more symptoms of narcolepsy. Methods of treating narcolepsy described herein also include administering to a patient in need thereof gaboxadol or a pharmaceutically acceptable salt thereof wherein the method provides improvement in next day functioning of the patient. In embodiments, a method of treating narcolepsy is provided which includes administering to a patient in need thereof gaboxadol or a pharmaceutically acceptable salt thereof, in combination with one or more of a CNS stimulant, a eugeroic agent, an antidepressant or a GABA_{B} receptor agonist. Methods of treating narcolepsy described herein also include administering to a patient in need thereof gaboxadol or a pharmaceutically acceptable salt thereof in combination with one or more of a CNS stimulant, a eugeroic agent, an antidepressant or a GABA_{B} receptor agonist, wherein the method provides improvement in one or more symptoms of narcolepsy. Methods of treating narcolepsy described herein also include administering to a patient in need thereof gaboxadol or a pharmaceutically acceptable salt thereof in combination with one or more of a CNS stimulant, a eugeroic agent, an antidepressant or a GABA_{B} receptor agonist, wherein the method provides improvement in next day functioning of the patient.

Symptoms of narcolepsy include excessive daytime sleepiness, abnormal REM sleep, cataplexy, sleep paralysis, hallucinations, automatic behaviors and night-time wakefulness.

Many pharmaceutical products are administered as a fixed dose, at regular intervals, to achieve therapeutic efficacy. The duration of action is reflected by a drug's plasma half-life. Gaboxadol is a selective GABA_{A} receptor agonist with a relatively short half-life (t½=1.5 h). Since efficacy is often dependent on sufficient exposure within the central nervous system administration of CNS drugs with a short half-life may require frequent maintenance dosing. Advantageously disclosed herein are methods of treating narcolepsy by administration of gaboxadol or a pharmaceutically acceptable salt thereof. For example, in embodiments, methods of treating narcolepsy are provided which include administering to a patient in need thereof a pharmaceutical composition including about 0.05 mg to about 30 mg gaboxadol or a pharmaceutically acceptable salt thereof wherein the composition provides improvement for more than 4 hours after administration to the patient. For example, in embodiments, methods of treating narcolepsy are provided which include administering to a patient in need thereof a pharmaceutical composition including about 1.0 mg to about 20 mg gaboxadol or a pharmaceutically acceptable salt thereof wherein the composition provides improvement for more than 6 hours after administration to the patient.

Embodiments described herein provide that a patient in need thereof is administered a pharmaceutical composition including gaboxadol or a pharmaceutically acceptable salt thereof. Gaboxadol or pharmaceutically acceptable salt thereof may be provided as an acid addition salt, a zwitter ion hydrate, zwitter ion anhydrate, hydrochloride or hydrobromide salt, or in the form of the zwitter ion monohydrate. Acid addition salts, include but are not limited to, maleic, fumaric, benzoic, ascorbic, succinic, oxalic, bis-methylenesalicylic, methanesulfonic, ethane-disulfonic, acetic, propionic, tartaric, salicylic, citric, gluconic, lactic, malic, mandelic, cinnamic, citraconic, aspartic, stearic, palmitic, itaconic, glycolic, p-amino-benzoic, glutamic, benzene sulfonic or theophylline acetic acid addition salts, as well as the 8-halotheophyllines, for example 8-bromo-theophylline. In other suitable embodiments, inorganic acid addition salts, including but not limited to, hydrochloric, hydrobromic, sulfuric, sulfamic, phosphoric or nitric acid addition salts may be used.

In embodiments, gaboxadol is provided as gaboxadol monohydrate. One skilled in the art will readily understand that the amounts of active ingredient in a pharmaceutical composition will depend on the form of gaboxadol provided. For example, pharmaceutical compositions of including 5.0, 10.0, or 15.0 mg gaboxadol correspond to 5.6, 11.3, or 16.9 mg gaboxadol monohydrate.

In embodiments, gaboxadol is crystalline, such as the crystalline hydrochloric acid salt, the crystalline hydrobromic acid salt, or the crystalline zwitter ion monohydrate. In embodiments, gaboxadol is provided as a crystalline monohydrate.

Deuteration of pharmaceuticals to improve pharmacokinetics (PK), pharmacodynamics (PD), and toxicity profiles, has been demonstrated previously with some classes of drugs. Accordingly the use of deuterium enriched gaboxadol is contemplated and within the scope of the methods and compositions described herein. Deuterium can be incorporated in any position in replace of hydrogen synthetically, according to the synthetic procedures known in the art. For example, deuterium may be incorporated to various positions having an exchangeable proton, such as the amine N—H, via proton-deuterium equilibrium exchange. Thus, deuterium may be incorporated selectively or non-selectively through methods known in the art to provide deuterium enriched gaboxadol. See Journal of Labeled Compounds and Radiopharmaceuticals 19(5) 689-702 (1982).

Deuterium enriched gaboxadol may be described by the percentage of incorporation of deuterium at a given position in the molecule in the place of hydrogen. For example, deuterium enrichment of 1% at a given position means that 1% of molecules in a given sample contain deuterium at that specified position. The deuterium enrichment can be determined using conventional analytical methods, such as mass spectrometry and nuclear magnetic resonance spectroscopy. In embodiments deuterium enriched gaboxadol means that the specified position is enriched with deuterium above the naturally occurring distribution (i.e., above about 0.0156%). In embodiments deuterium enrichment is no less than about 1%, no less than about 5%, no less than about 10%, no less than about 20%, no less than about 50%, no less than about 70%, no less than about 80%, no less than about 90%, or no less than about 98% of deuterium at a specified position.

In embodiments, methods of treating narcolepsy include administering to a patient in need thereof a pharmaceutical composition including about 0.05 mg to about 50 mg gaboxadol or a pharmaceutically acceptable salt thereof. Pharmaceutical compositions (also referred to herein as “pharmaceutical formulations” or simply “formulations”) herein encompass dosage forms. Dosage forms herein encompass unit doses. In embodiments, as discussed below, various dosage forms including conventional formulations and modified release formulations can be administered one or more times daily. Any suitable route of administration may be utilized, e.g., oral, rectal, nasal, pulmonary, vaginal, sublingual, transdermal, intravenous, intraarterial, intramuscular, intraperitoneal and subcutaneous routes. Suitable dosage forms include tablets, capsules, oral liquids, powders, aerosols, transdermal modalities such as topical liquids, patches, creams and ointments, parenteral formulations and suppositories.

In embodiments, the pharmaceutical compositions include 0.1 to 50 mg, 0.1 mg to 25 mg, 0.1 mg to 20 mg, 0.1 mg to 15 mg, 0.5 mg to 25 mg, 0.5 mg to 20 mg, 0.5 to 15 mg, 1 mg to 50 mg, 1 mg to 25 mg, 1 mg to 20 mg, 1 mg to 15 mg, 1.5 mg to 25 mg, 1.5 mg to 20 mg, 1.5 mg to 15 mg, 2 mg to 25 mg, 2 mg to 20 mg, 2 mg to 15 mg, 2.5 mg to 30 mg, 2.5 mg to 25 mg, 2.5 mg to 20 mg, 2.5 mg to 15 mg, 3 mg to 25 mg, 3 mg to 20 mg, 3 mg to 15 mg gaboxadol or a pharmaceutically acceptable salt thereof.

In embodiments, the pharmaceutical compositions include 5 mg to 20 mg, 5 mg to 10 mg, 4 mg to 6 mg, 6 mg to 8 mg, 8 mg to 10 mg, 10 mg to 12 mg, 12 mg to 14 mg, 14 mg to 16 mg, 16 mg to 18 mg, or 18 mg to 20 mg gaboxadol or a pharmaceutically acceptable salt thereof.

In embodiments, the pharmaceutical compositions include 0.1 mg, 0.25 mg, 0.5 mg, 1 mg, 2.5 mg, 3 mg, 4 mg, 5 mg, 6 mg, 7 mg, 7.5 mg, 8 mg, 9 mg, 10 mg, 11 mg, 12 mg, 12.5 mg, 13 mg, 14 mg, 15 mg, 16 mg, 17 mg, 17.5 mg, 18 mg, 19 mg, 20 mg, 21 mg, 22 mg, 23 mg, 24 mg, 25 mg, 26 mg, 27 mg, 28 mg, 29 mg, or 30 mg gaboxadol or a pharmaceutically acceptable salt thereof or amounts that are multiples of such doses. In embodiments, the pharmaceutical compositions include 2.5 mg, 5 mg, 7.5 mg, 10 mg, 15 mg, or 20 mg gaboxadol or a pharmaceutically acceptable salt thereof.

In embodiments, the pharmaceutical compositions described herein are administered once, twice, or three times daily, or every other day. In embodiments, a pharmaceutical composition described herein is provided to the patient in the morning. In embodiments, a pharmaceutical composition described herein is provided to the patient in the evening. In embodiments, a pharmaceutical composition described herein is provided to the patient once in the evening and once in the morning. In embodiments, a pharmaceutical composition described herein is administered to the patient at bedtime.

In embodiments, the total amount of gaboxadol or a pharmaceutically acceptable salt thereof administered to a subject in a 24-hour period is 1 mg to 50 mg. In embodiments, the total amount of gaboxadol or a pharmaceutically acceptable salt thereof administered to a subject in a 24-hour period is 1 mg to 30 mg. In embodiments, the total amount of gaboxadol or a pharmaceutically acceptable salt thereof administered to a subject in a 24-hour period is 1 mg to 20 mg. In embodiments, the total amount of gaboxadol or a pharmaceutically acceptable salt thereof administered to a subject in a 24-hour period is 1 mg to 15 mg. In embodiments, the total amount of gaboxadol or a pharmaceutically acceptable salt thereof administered to a subject in a 24-hour period is 1 mg to 10 mg. In embodiments, the total amount of gaboxadol or a pharmaceutically acceptable salt thereof administered to a subject in a 24-hour period is 1 mg to 5 mg. In embodiments, the total amount of gaboxadol or a pharmaceutically acceptable salt thereof administered to a subject in a 24-hour period is 5 mg, 10 mg, or 15 mg. In embodiments, the total amount of gaboxadol or a pharmaceutically acceptable salt thereof administered to a subject in a 24-hour period is 20 mg. In embodiments, the total amount of gaboxadol or a pharmaceutically acceptable salt thereof administered to a subject in a 24-hour period is 25 mg.

In embodiments, a patient is administered gaboxadol or a pharmaceutically acceptable salt thereof in an amount ranging from 0.1 mg/kg to 3.0 mg/kg.

In embodiments, provided herein are methods of treating narcolepsy including administering to a patient in need thereof a pharmaceutical composition including gaboxadol or a pharmaceutically acceptable salt thereof wherein the composition provides improvement in at least one symptom of narcolepsy.

In embodiments, provided herein are methods of treating narcolepsy including administering to a patient in need thereof a pharmaceutical composition including gaboxadol or a pharmaceutically acceptable salt thereof wherein the composition provides improvement of at least one symptom for more than 4 hours after administration of the pharmaceutical composition to the patient. In embodiments, the improvement of at least one symptom for more than 6 hours after administration of the pharmaceutical composition to the patient is provided in accordance with the present disclosure. In embodiments, improvement of at least one symptom for more than, e.g., 8 hours, 10 hours, 12 hours, 15 hours, 18 hours, 20 hours, or 24 hours after administration of the pharmaceutical composition to the patient is provided in accordance with the present disclosure. In embodiments, improvement in at least one symptom for at least e.g., 8 hours, 10 hours, 12 hours, 15 hours, 18 hours, 20 hours, or 24 hours after administration of the pharmaceutical composition to the patient is provided in accordance with the present disclosure. In embodiments, improvement in at least one symptom for 12 hours after administration of the pharmaceutical composition to the patient is provided in accordance with the present disclosure.

In embodiments, provided herein methods of treating narcolepsy including administering to a patient in need thereof a pharmaceutical composition including gaboxadol or a pharmaceutically acceptable salt thereof wherein the composition provides improvement in next day functioning to the patient.

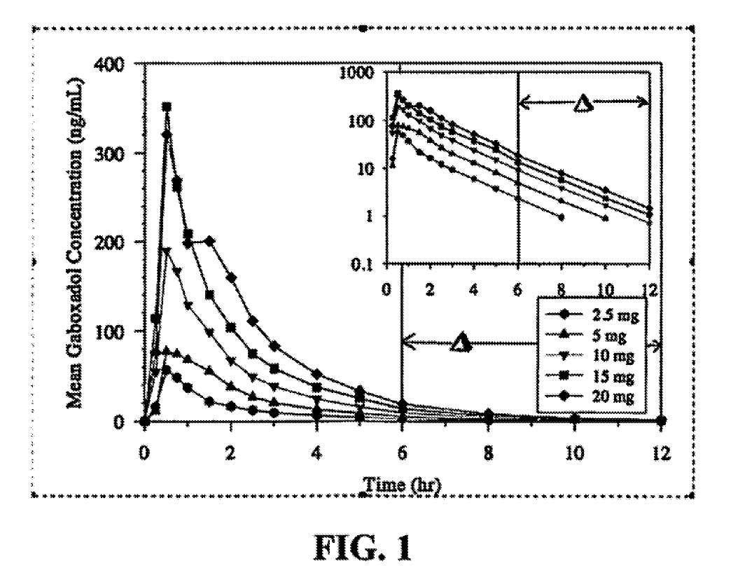

FIG. 1 shows the arithmetic mean plasma concentration-time profiles of gaboxadol following single oral doses (2.5, 5, 10, 15, and 20 mg)(see, Example 1, below) with horizontal lines Δ indicating the change between 6 and 12 hours. In embodiments, provided herein are methods of treating narcolepsy including administering to a patient in need thereof about 0.05 mg to about 30 mg gaboxadol or a pharmaceutically acceptable salt thereof which provides an in vivo plasma profile, wherein the in vivo plasma profile of the patient 6 hours after administration of the gaboxadol or pharmaceutically acceptable salt thereof is reduced by more than 50% and the method provides improvement in the patient for more than 6, 8, 10, 12, 14, 16, 18, 20, 22 or 24 hours after administration. In embodiments, provided herein are methods of treating narcolepsy including administering to a patient in need thereof about 0.05 mg to about 30 mg gaboxadol or a pharmaceutically acceptable salt thereof which provides an in vivo plasma profile, wherein the in vivo plasma profile of the patient 6 hours after administration of the gaboxadol or pharmaceutically acceptable salt thereof is reduced by more than 55% and the method provides improvement in the patient for more than 6, 8, 10, 12, 14, 16, 18, 20, 22 or 24 hours after administration. In embodiments, provided herein are methods of treating narcolepsy including administering to a patient in need thereof about 0.05 mg to about 30 mg gaboxadol or a pharmaceutically acceptable salt thereof which provides an in vivo plasma profile, wherein the in vivo plasma profile of the patient 6 hours after administration of the gaboxadol or pharmaceutically acceptable salt thereof is reduced by more than 60% and the method provides improvement in the patient for more than 6, 8, 10, 12, 14, 16, 18, 20, 22 or 24 hours after administration. In embodiments, provided herein are methods of treating narcolepsy including administering to a patient in need thereof about 0.05 mg to about 30 mg gaboxadol or a pharmaceutically acceptable salt thereof which provides an in vivo plasma profile, wherein the in vivo plasma profile of the patient 6 hours after administration of the gaboxadol or pharmaceutically acceptable salt thereof is reduced by more than 65% and the method provides improvement in the patient for more than 6, 8, 10, 12, 14, 16, 18, 20, 22 or 24 hours after administration. In embodiments, provided herein are methods of treating narcolepsy including administering to a patient in need thereof about 0.05 mg to about 30 mg gaboxadol or a pharmaceutically acceptable salt thereof which provides an in vivo plasma profile, wherein the in vivo plasma profile of the patient 6 hours after administration of the gaboxadol or pharmaceutically acceptable salt thereof is reduced by more than 70% and the method provides improvement in the patient for more than 6, 8, 10, 12, 14, 16, 18, 20, 22 or 24 hours after administration. In embodiments, provided herein are methods of treating narcolepsy including administering to a patient in need thereof about 0.05 mg to about 30 mg gaboxadol or a pharmaceutically acceptable salt thereof which provides an in vivo plasma profile, wherein the in vivo plasma profile of the patient 6 hours after administration of the gaboxadol or pharmaceutically acceptable salt thereof is reduced by more than 75% and the method provides improvement in the patient for more than 6, 8, 10, 12, 14, 16, 18, 20, 22 or 24 hours after administration.

In embodiments, provided herein are methods of treating narcolepsy wherein the amount of gaboxadol or pharmaceutically acceptable salt thereof within the patient about 4 hours after administration of the pharmaceutical composition is less than about 75% of the administered dose. In embodiments, provided herein are methods wherein the amount of gaboxadol or pharmaceutically acceptable salt thereof within the patient about, e.g., 6 hours, 8 hours, 10 hours, 12 hours, 15 hours, or 20 hours after administration of the pharmaceutical composition is less than about 75%.

In embodiments, provided herein are methods of treating narcolepsy wherein the amount of gaboxadol or pharmaceutically acceptable salt thereof within the patient about 4 hours after administration of the pharmaceutical composition is less than about 80% of the administered dose. In embodiments, provided herein are methods wherein the amount of gaboxadol or pharmaceutically acceptable salt thereof within the patient about, e.g., 6 hours, 8 hours, 10 hours, 12 hours, 15 hours, or 20 hours after administration of the pharmaceutical composition is less than about 80% of the administered dose.

In embodiments, provided herein are methods of treating narcolepsy wherein the amount of gaboxadol or pharmaceutically acceptable salt thereof within the patient about 4 hours after administration of the pharmaceutical composition is between about 65% to about 85% of the administered dose. In embodiments, the amount of gaboxadol or pharmaceutically acceptable salt thereof within the patient after about, e.g., 6 hours, 8 hours, 10 hours, 12 hours, 15 hours, or 20 hours after administration of the pharmaceutical composition is between about 65% to about 85% of the administered dose.

In embodiments, provided herein are methods of treating narcolepsy including administering to a patient in need thereof a pharmaceutical composition including gaboxadol or a pharmaceutically acceptable salt thereof wherein the composition provides an in vivo plasma concentration 6 hours after administration which is less than 75% of the administered dose and provides improvement in the patient for more than 6, 8, 10, 12, 14, 16, 18, 20, 22 or 24 hours after administration. In embodiments, provided herein are methods of treating narcolepsy including administering to a patient in need thereof a pharmaceutical composition including gaboxadol or a pharmaceutically acceptable salt thereof wherein the composition provides an in vivo plasma concentration 6 hours after administration which is less than 80% of the administered dose and provides improvement in the patient for more than 6, 8, 10, 12, 14, 16, 18, 20, 22 or 24 hours after administration. In embodiments, provided herein are methods of treating narcolepsy including administering to a patient in need thereof a pharmaceutical composition including gaboxadol or a pharmaceutically acceptable salt thereof wherein the composition provides an in vivo plasma concentration 6 hours after administration which is less than 85% of the administered dose and provides improvement in the patient for more than 6, 8, 10, 12, 14, 16, 18, 20, 22 or 24 hours after administration. In embodiments, provided herein are methods of treating narcolepsy including administering to a patient in need thereof a pharmaceutical composition including gaboxadol or a pharmaceutically acceptable salt thereof wherein the composition provides an in vivo plasma concentration 6 hours after administration which is less than 90% of the administered dose and provides improvement in the patient for more than 6, 8, 10, 12, 14, 16, 18, 20, 22 or 24 hours after administration. In embodiments, provided herein are methods of treating narcolepsy including administering to a patient in need thereof a pharmaceutical composition including gaboxadol or a pharmaceutically acceptable salt thereof wherein the composition provides an in vivo plasma concentration 6 hours after administration which is less than 95% of the administered dose and provides improvement in the patient for more than 6, 8, 10, 12, 14, 16, 18, 20, 22 or 24 hours after administration. In embodiments, provided herein are methods of treating narcolepsy including administering to a patient in need thereof a pharmaceutical composition including gaboxadol or a pharmaceutically acceptable salt thereof wherein the composition provides an in vivo plasma concentration 6 hours after administration which is less than 100% of the administered dose and provides improvement in the patient for more than 6, 8, 10, 12, 14, 16, 18, 20, 22 or 24 hours after administration.

In embodiments, provided herein are methods of treating narcolepsy including administering to a patient in need thereof a pharmaceutical composition including gaboxadol or a pharmaceutically acceptable salt thereof wherein the composition provides an in vivo plasma profile having a Cₘₐₓ less than about 500 ng/ml. In embodiments, the composition provides improvement for more than 6 hours after administration to the patient.

In embodiments, the composition provides an in vivo plasma profile having a Cₘₐₓ less than about, e.g., 450 ng/ml, 400 ng/ml 350 ng/ml, or 300 ng/ml and wherein the composition provides improvement of next day functioning of the patient. In embodiments, the composition provides an in vivo plasma profile having a Cₘₐₓ less than about, e.g., 250 ng/ml, 200 ng/ml 150 ng/ml, or 100 ng/ml and wherein the composition provides improvement of next day functioning of the patient.

In embodiments, provided herein are methods of treating narcolepsy including administering to a patient in need thereof a pharmaceutical composition including gaboxadol or a pharmaceutically acceptable salt thereof wherein the composition provides an in vivo plasma profile having a AUC_{0-∞} of less than about 900 ng·hr/ml. In embodiments, the composition provides improvement in next day functioning of the patient. In embodiments, the compositions provide an in vivo plasma profile having a AUC_{0-∞} of less than about, e.g., 850 ng·hr/ml, 800 ng·hr/ml, 750 ng·hr/ml, or 700 ng·hr/ml and wherein the composition provides improvement of next day functioning of the patient. In embodiments, the composition provides improvement in one or more symptom for more than 6 hours after administration.

In embodiments, provided herein are methods of treating narcolepsy including administering to a patient in need thereof a pharmaceutical composition including gaboxadol or a pharmaceutically acceptable salt thereof wherein the composition provides an in vivo plasma profile having a AUC_{0-∞} of less than about, e.g., 650 ng·hr/ml, 600 ng·hr/ml, 550 ng·hr/ml, 500 ng·hr/ml, or 450 ng·hr/ml. In embodiments, wherein the composition provides an in vivo plasma profile having a AUC_{0-∞} of less than about, e.g., 400 ng·hr/ml, 350 ng·hr/ml, 300 ng·hr/ml, 250 ng·hr/ml, or 200 ng·hr/ml. In embodiments, the composition provides an in vivo plasma profile having a AUC_{0-∞} of less than about, e.g., 150 ng·hr/ml, 100 ng·hr/ml, 75 ng·hr/ml, or 50 ng·hr/ml. In embodiments, the composition provides improvement of next day functioning of the patient after administration for more than, e.g., 4 hours, 6 hours, 8 hours, 10 hours, or 12 hours, after administration of the composition to the patient.

In embodiments, provided herein are methods of treating narcolepsy including administering to a patient in need thereof an amount of gaboxadol or a pharmaceutically acceptable salt thereof which provides an in vivo plasma profile having a AUC₆₋₁₂ which is less than 75% of the Cₘₐₓ and provides improvement in the patient for more than 6, 8, 10, 12, 14, 16, 18, 20, 22 or 24 hours after administration. In embodiments, provided herein are methods of treating narcolepsy including administering to a patient in need thereof an amount of gaboxadol or a pharmaceutically acceptable salt thereof which provides an in vivo plasma profile having a AUC₆₋₁₂ which is less than 80% of the Cₘₐₓ and provides improvement in the patient for more than 6, 8, 10, 12, 14, 16, 18, 20, 22 or 24 hours after administration. In embodiments, provided herein are methods of treating narcolepsy including administering to a patient in need thereof an amount of gaboxadol or a pharmaceutically acceptable salt thereof which provides an in vivo plasma profile having a AUC₆₋₁₂ which is less than 85% of the Cₘₐₓ and provides improvement in the patient for more than 6, 8, 10, 12, 14, 16, 18, 20, 22 or 24 hours after administration. In embodiments, provided herein are methods of treating narcolepsy including administering to a patient in need thereof an amount of gaboxadol or a pharmaceutically acceptable salt thereof which provides an in vivo plasma profile having a AUC₆₋₁₂ which is less than 90% of the Cₘₐₓ and provides improvement in the patient for more than 6, 8, 10, 12, 14, 16, 18, 20, 22 or 24 hours after administration. In embodiments, provided herein are methods of treating narcolepsy including administering to a patient in need thereof an amount of gaboxadol or a pharmaceutically acceptable salt thereof which provides an in vivo plasma profile having a AUC₆₋₁₂ which is less than 95% of the Cₘₐₓ and provides improvement in the patient for more than 6, 8, 10, 12, 14, 16, 18, 20, 22 or 24 hours after administration. In embodiments, provided herein are methods of treating narcolepsy including administering to a patient in need thereof an amount of gaboxadol or a pharmaceutically acceptable salt thereof which provides an in vivo plasma profile having a AUC₆₋₁₂ which is less than 100% of the Cₘₐₓ and provides improvement in the patient for more than 6, 8, 10, 12, 14, 16, 18, 20, 22 or 24 hours after administration.

In embodiments, provided herein are methods of treating narcolepsy including administering to a patient in need thereof a pharmaceutical composition including gaboxadol or a pharmaceutically acceptable salt thereof wherein the composition provides an in vivo plasma profile having a AUC₆₋₁₂ which is less than 75% of the Cₘₐₓ and provides improvement in the patient for more than 6, 8, 10, 12, 14, 16, 18, 20, 22 or 24 hours after administration. In embodiments, provided herein are methods of treating narcolepsy including administering to a patient in need thereof a pharmaceutical composition including gaboxadol or a pharmaceutically acceptable salt thereof wherein the composition provides an in vivo plasma profile having a AUC₆₋₁₂ which is less than 80% of the Cₘₐₓ and provides improvement in the patient for more than 6, 8, 10, 12, 14, 16, 18, 20, 22 or 24 hours after administration. In embodiments, provided herein are methods of treating narcolepsy including administering to a patient in need thereof a pharmaceutical composition including gaboxadol or a pharmaceutically acceptable salt thereof wherein the composition provides an in vivo plasma profile having a AUC₆₋₁₂ which is less than 85% of the Cₘₐₓ and provides improvement in the patient for more than 6, 8, 10, 12, 14, 16, 18, 20, 22 or 24 hours after administration. In embodiments, provided herein are methods of treating narcolepsy including administering to a patient in need thereof a pharmaceutical composition including gaboxadol or a pharmaceutically acceptable salt thereof wherein the composition provides an in vivo plasma profile having a AUC₆₋₁₂ which is less than 90% of the Cₘₐₓ and provides improvement in the patient for more than 6, 8, 10, 12, 14, 16, 18, 20, 22 or 24 hours after administration. In embodiments, provided herein are methods of treating narcolepsy including administering to a patient in need thereof a pharmaceutical composition including gaboxadol or a pharmaceutically acceptable salt thereof wherein the composition provides an in vivo plasma profile having a AUC₆₋₁₂ which is less than 95% of the Cₘₐₓ and provides improvement in the patient for more than 6, 8, 10, 12, 14, 16, 18, 20, 22 or 24 hours after administration. In embodiments, provided herein are methods of treating narcolepsy including administering to a patient in need thereof a pharmaceutical composition including gaboxadol or a pharmaceutically acceptable salt thereof wherein the composition provides an in vivo plasma profile having a AUC₆₋₁₂ which is less than 100% of the Cₘₐₓ and provides improvement in the patient for more than 6, 8, 10, 12, 14, 16, 18, 20, 22 or 24 hours after administration.

In embodiments, provided herein are methods of treating narcolepsy including administering to a patient in need thereof a pharmaceutical composition including gaboxadol or a pharmaceutically acceptable salt thereof wherein the composition provides an in vivo plasma profile having a AUC₆₋₁₂ which is less than 75% of the administered dose and provides improvement in the patient for more than 6, 8, 10, 12, 14, 16, 18, 20, 22 or 24 hours after administration. In embodiments, provided herein are methods of treating narcolepsy including administering to a patient in need thereof a pharmaceutical composition including gaboxadol or a pharmaceutically acceptable salt thereof wherein the composition provides an in vivo plasma profile having a AUC₆₋₁₂ which is less than 80% of the administered dose and provides improvement in the patient for more than 6, 8, 10, 12, 14, 16, 18, 20, 22 or 24 hours after administration. In embodiments, provided herein are methods of treating narcolepsy including administering to a patient in need thereof a pharmaceutical composition including gaboxadol or a pharmaceutically acceptable salt thereof wherein the composition provides an in vivo plasma profile having a AUC₆₋₁₂ which is less than 85% of the administered dose and provides improvement in the patient for more than 6, 8, 10, 12, 14, 16, 18, 20, 22 or 24 hours after administration. In embodiments, provided herein are methods of treating narcolepsy including administering to a patient in need thereof a pharmaceutical composition including gaboxadol or a pharmaceutically acceptable salt thereof wherein the composition provides an in vivo plasma profile having a AUC₆₋₁₂ which is less than 90% of the administered dose and provides improvement in the patient for more than 6, 8, 10, 12, 14, 16, 18, 20, 22 or 24 hours after administration. In embodiments, provided herein are methods of treating narcolepsy including administering to a patient in need thereof a pharmaceutical composition including gaboxadol or a pharmaceutically acceptable salt thereof wherein the composition provides an in vivo plasma profile having a AUC₆₋₁₂ which is less than 95% of the administered dose and provides improvement in the patient for more than 6, 8, 10, 12, 14, 16, 18, 20, 22 or 24 hours after administration. In embodiments, provided herein are methods of treating narcolepsy including administering to a patient in need thereof a pharmaceutical composition including gaboxadol or a pharmaceutically acceptable salt thereof wherein the composition provides an in vivo plasma profile having a AUC₆₋₁₂ which is less than 100% of the administered dose and provides improvement in the patient for more than 6, 8, 10, 12, 14, 16, 18, 20, 22 or 24 hours after administration.

In embodiments, provided herein are methods of treating narcolepsy including administering to a patient in need thereof a first pharmaceutical composition including gaboxadol or a pharmaceutically acceptable salt thereof and a second pharmaceutical composition including gaboxadol or a pharmaceutically acceptable salt thereof wherein the second pharmaceutical composition provides an in vivo plasma profile having a mean AUC_{0-∞} of at least about 20% less than the first pharmaceutical composition.

In embodiments the first and/or the second pharmaceutical compositions are administered once, twice, or three times daily, or every other day. In embodiments, the first or the second pharmaceutical composition is provided to the patient in the evening. In embodiments, the first or the second pharmaceutical composition is provided to the patient in the morning. In embodiments, the second pharmaceutical composition includes an amount of gaboxadol that is at least one third of the amount of gaboxadol provided in the first pharmaceutical composition. In embodiments, the second pharmaceutical composition includes an amount of gaboxadol that is at least half of the amount of gaboxadol provided in the first pharmaceutical composition.

In embodiments, the first or the second pharmaceutical composition is provided to the patient once in the evening and once in the morning. In embodiments, the total amount of gaboxadol or pharmaceutically acceptable salt thereof administered to a subject in a 24-hour period is 1 mg to 30 mg. In embodiments, the total amount of gaboxadol or a pharmaceutically acceptable salt thereof administered to a subject in a 24-hour period is 1 mg to 20 mg. In embodiments, the total amount of gaboxadol or a pharmaceutically acceptable salt thereof administered to a subject in a 24-hour period is 10 mg, 15 mg, or 20 mg. In embodiments, the total amount of gaboxadol or a pharmaceutically acceptable salt thereof administered to a subject in a 24-hour period is 20 mg.

In embodiments, the first and/or the second pharmaceutical compositions may be provided with conventional release or modified release profiles which include delayed release or extended release profiles. The first and second pharmaceutical compositions may be provided at the same time or separated by an interval of time, e.g., 6 hours, 12 hours etc. In embodiments, the first and the second pharmaceutical compositions may be provided with different drug release profiles to create a two-phase release profile. For example, the first pharmaceutical composition may be provided with an immediate release profile and the second pharmaceutical composition may provide an extended release profile. In embodiments, one or both of the first and second pharmaceutical compositions may be provided with an extended release or delayed release profile. Such compositions may be provided as pulsatile formulations, multilayer tablets or capsules containing tablets, beads, granules, etc. In embodiments, the first pharmaceutical composition is an immediate release composition. In embodiments, the second pharmaceutical composition is an immediate release composition. In embodiments, the first and second pharmaceutical compositions are provided as separate immediate release compositions, e.g., tablets or capsules. In embodiments the first and second pharmaceutical compositions are provided 12 hours apart.

In embodiments, provided herein are methods of treating narcolepsy including administering to a patient in need thereof a first pharmaceutical composition including gaboxadol or a pharmaceutically acceptable salt thereof and a second pharmaceutical composition including gaboxadol or a pharmaceutically acceptable salt thereof wherein the second pharmaceutical composition provides an in vivo plasma profile having a mean AUC_{0-∞} of at least about, e.g., 25%, 30%, 35%, 40%, 45% or 50% less than the first pharmaceutical composition. In embodiments, the composition provides improvement of next day functioning of the patient. For example, the composition may provide improvement in one or more symptoms for more than about, e.g., 6 hours, 8 hours, 10 hours, or 12 hours after administration of the first and/or second pharmaceutical composition.

In embodiments, provided herein are methods of treating narcolepsy including administering to a patient in need thereof a first pharmaceutical composition including gaboxadol or a pharmaceutically acceptable salt thereof and a second pharmaceutical composition including gaboxadol or a pharmaceutically acceptable salt thereof wherein the second pharmaceutical composition provides an in vivo plasma profile having a mean AUG_{0-∞} of less than about 900 ng·hr/ml. In embodiments, the second pharmaceutical composition provides an in vivo plasma profile having a AUC_{0-∞} of less than about, e.g., 800 ng·hr/ml, 750 ng·hr/ml, 700 ng·hr/ml, 650 ng·hr/ml, or 600 ng·hr/ml. In embodiments, the second pharmaceutical composition provides an in vivo plasma profile having a AUC_{0-∞} of less than about, e.g., 550 ng·hr/ml, 500 ng·hr/ml, 450 ng·hr/ml, 400 ng·hr/ml, or 350 ng·hr/ml. In embodiments, the second pharmaceutical composition provides an in vivo plasma profile having a AUC_{0-∞} of less than about, e.g., 300 ng·hr/ml, 250 ng·hr/ml, 200 ng·hr/ml, 150 ng·hr/ml, or 100 ng·hr/ml. In embodiments, the first and second pharmaceutical composition are administered wherein the compositions provide improvement of next day functioning of the patient. In embodiments, the first pharmaceutical composition provides improvement in one or more symptom for more than, e.g., 6 hours, 8 hours or 12 hours after administration of the first pharmaceutical composition.

In embodiments, provided herein are methods of treating narcolepsy including administering to a patient in need thereof a first pharmaceutical composition including gaboxadol or a pharmaceutically acceptable salt thereof and a second pharmaceutical composition including gaboxadol or a pharmaceutically acceptable salt thereof wherein the first composition provides an in vivo plasma profile with a Cₘₐₓ that is more than about 50% greater than the Cₘₐₓ provided by the administration of the second pharmaceutical composition. As used herein the Cₘₐₓ provided by the administration of the second pharmaceutical composition may or may not include the plasma profile contribution of the first pharmaceutical composition. In embodiments, the administration of the second pharmaceutical composition does not include the plasma profile contribution of the first pharmaceutical composition. In embodiments, the first composition provides an in vivo plasma profile having a Cₘₐₓ that is more than about e.g., 60%, 70%, 80%, or 90% greater than the Cₘₐₓ provided by the administration of the second pharmaceutical composition.

In embodiments, the Tₘₐₓ of the first pharmaceutical composition is less than 3 hours. In embodiments, the Tₘₐₓ of the first pharmaceutical composition is less than 2.5 hours. In embodiments, the Tₘₐₓ of the first pharmaceutical composition is less than 2 hours. In embodiments, the Tₘₐₓ of the first pharmaceutical composition is less than 1.5 hours. In embodiments, the Tₘₐₓ of the first pharmaceutical composition is less than 1 hour.

In embodiments, the first pharmaceutical composition provides a dissolution of at least about 80% within the first 20 minutes of administration to a patient in need thereof. In embodiments, the first pharmaceutical composition provides a dissolution of at least about, e.g., 85%, 90% or 95% within the first 20 minutes of administration to a patient in need thereof. In embodiments, the first pharmaceutical composition provides a dissolution of at least 80% within the first 10 minutes of administration to a patient in need thereof.

In embodiments the first and/or the second pharmaceutical compositions are sub therapeutic dosages. A sub therapeutic dosage is an amount of active agent such as gaboxadol pharmaceutically acceptable salt thereof that is less than the amount required for a therapeutic effect. In embodiments, a sub therapeutic dosage is an amount of active agent such as gaboxadol or a pharmaceutically acceptable salt thereof that alone may not provide improvement in at least one symptom of narcolepsy but is sufficient to maintain such improvement. In embodiments, the methods provide administering a first pharmaceutical composition that provides improvement in at least one symptom of narcolepsy and a second composition that maintains the improvement. In embodiments, after administration of the first pharmaceutical composition the second pharmaceutical composition may provide a synergistic effect to improve at least one symptom of narcolepsy. In embodiments the second pharmaceutical composition may provide a synergistic effect to improve at least one symptom of narcolepsy.

In embodiments, provided herein are methods of treating narcolepsy including administering to a patient in need thereof a pharmaceutical composition including a first pharmaceutical dosage including gaboxadol or a pharmaceutically acceptable salt thereof wherein the composition provides improvement for more than 6 hours after administration and a second pharmaceutical composition including a sub therapeutic dosage of gaboxadol or a pharmaceutically acceptable salt thereof.

Administration of the first and second pharmaceutical compositions may be separated by an interval of time to achieve long-term improvement in at least one symptom. In embodiments, the first and second pharmaceutical composition may be administered 6 hours apart. In embodiments the first and second pharmaceutical composition may be administered 12 hours apart. In embodiments, the first and second pharmaceutical compositions may administered within, e.g., 6 hours, 12 hours, 18 hours, 24 hours etc. In embodiments, the first and second pharmaceutical compositions may administered separated by at least, e.g., 6 hours, 12 hours, 18 hours, 24 hours etc. In embodiments, improvement in at least one symptom of narcolepsy for more than 8 hours after administration to the patient is provided. In embodiments, improvement for more than about, e.g., 10 hours, 12 hours, 15 hours, 18 hours, 20 hours, or 24 hours after administration to the patient is provided.

In embodiments, the first pharmaceutical composition and/or the second pharmaceutical composition include about 0.1 mg to about 40 mg gaboxadol or a pharmaceutically acceptable salt thereof. The amount of gaboxadol or a pharmaceutically acceptable salt thereof in the first pharmaceutical composition and the second pharmaceutical composition may be the same or different. In embodiments, the administration of the first and second pharmaceutical composition may provide a synergistic effect to improve at least one symptom of narcolepsy.

In embodiments, the first and/or the second pharmaceutical composition include 0.1 mg to 25 mg, 0.1 mg to 20 mg, 0.1 mg to 15 mg, 0.5 mg to 25 mg, 0.5 mg to 20 mg, 0.5 to 15 mg, 1 mg to 25 mg, 1 mg to 20 mg, 1 mg to 15 mg, 1.5 mg to 25 mg, 1.5 mg to 20 mg, 1.5 mg to 15 mg, 2 mg to 25 mg, 2 mg to 20 mg, 2 mg to 15 mg, 2.5 mg to 25 mg, 2.5 mg to 20 mg, 2.5 mg to 15 mg, 3 mg to 25 mg, 3 mg to 20 mg, or 3 mg to 15 mg gaboxadol or a pharmaceutically acceptable salt thereof.

In embodiments, the first and/or the second pharmaceutical composition include 5 mg to 15 mg, 5 mg to 10 mg, 4 mg to 6 mg, 6 mg to 8 mg, 8 mg to 10 mg, 10 mg to 12 mg, 12 mg to 14 mg, 14 mg to 16 mg, 16 mg to 18 mg, or 18 mg to 20 mg gaboxadol or a pharmaceutically acceptable salt thereof.

In embodiments, the first and/or the second pharmaceutical composition include 0.1 mg, 0.25 mg, 0.5 mg, 1 mg, 2.5 mg, 3 mg, 4 mg, 5 mg, 6 mg, 7 mg, 7.5 mg, 8 mg, 9 mg, 10 mg, 11 mg, 12 mg, 12.5 mg, 13 mg, 14 mg, 15 mg, 16 mg, 17 mg, 17.5 mg, 18 mg, 19 mg, 20 mg, 21 mg, 22 mg, 23 mg, 24 mg, 25 mg, 26 mg, 27 mg, 28 mg, 29 mg, or 30 mg gaboxadol or a pharmaceutically acceptable salt thereof or amounts that are multiples of such doses. In embodiments, the first pharmaceutical compositions include 2.5 mg, 5 mg, 7.5 mg, 10 mg, 15 mg, or 20 mg gaboxadol or a pharmaceutically acceptable salt thereof. In embodiments, the second pharmaceutical compositions include 2.5 mg, 5 mg, 7.5 mg, 10 mg, 15 mg, or 20 mg gaboxadol or a pharmaceutically acceptable salt thereof.

Also provided herein is a method of treating narcolepsy which includes administering to a patient in need thereof gaboxadol or a pharmaceutically acceptable salt thereof, in combination with one or more of a CNS stimulant, a eugeroic agent, an antidepressant or a GABA_{B} receptor agonist. In embodiments, methods of treating narcolepsy are provided which include administering to a patient in need thereof gaboxadol or a pharmaceutical salt thereof, in combination with one or more of a CNS stimulant, a eugeroic agent, an antidepressant or a GABA_{B} receptor agonist, wherein the patient exhibits improvement in one or more symptoms of narcolepsy. In embodiments, methods of treating narcolepsy are provided which include administering to a patient in need thereof gaboxadol or a pharmaceutical salt thereof, in combination with one or more of a CNS stimulant, a eugeroic agent, an antidepressant or a GABA_{B} receptor agonist, in an amount effective to provide the patient with improvement in one or more symptoms of narcolepsy. In embodiments, methods of treating narcolepsy are provided which include administering to a patient in need thereof gaboxadol or a pharmaceutical salt thereof, in combination with one or more of a CNS stimulant, a eugeroic agent, an antidepressant or a GABA_{B} receptor agonist, wherein the patient exhibits improvement in one or more symptoms of narcolepsy for more than 4 hours after administration to the patient. In embodiments, methods of treating narcolepsy are provided which include administering to a patient in need thereof gaboxadol or a pharmaceutical salt thereof, in combination with one or more of a CNS stimulant, a eugeroic agent, an antidepressant or a GABA_{B} receptor agonist an amount effective to provide the patient with improvement in one or more symptoms of narcolepsy for more than 4 hours after administration to the patient. In embodiments, methods of treating narcolepsy are provided which include administering to a patient in need thereof gaboxadol or a pharmaceutical salt thereof, in combination with one or more of a CNS stimulant, a eugeroic agent, an antidepressant or a GABA_{B} receptor agonist, wherein the patient exhibits improvement in one or more symptoms of narcolepsy for more than 6 hours after administration to the patient. In embodiments, methods of treating narcolepsy are provided which include administering to a patient in need thereof gaboxadol or a pharmaceutical salt thereof, in combination with one or more of a CNS stimulant, a eugeroic agent, an antidepressant or a GABA_{B} receptor agonist an amount effective to provide the patient with improvement in one or more symptoms of narcolepsy for more than 6 hours after administration to the patient. In embodiments, methods of treating narcolepsy are provided which include administering to a patient in need thereof gaboxadol or a pharmaceutical salt thereof, in combination with one or more of a CNS stimulant, a eugeroic agent, an antidepressant or a GABA_{B} receptor agonist, wherein improvement in one or more symptoms of narcolepsy occurs for more than 8, 10, 12, 14, 16, 18, 20, 22 or 24 hours after administration to the patient.

In embodiments, methods of treating narcolepsy are provided which include administering to a patient in need thereof a pharmaceutical composition including gaboxadol or a pharmaceutical salt thereof, in combination with one or more of a CNS stimulant, a eugeroic agent, an antidepressant or a GABA_{B} receptor agonist. In embodiments, methods of treating narcolepsy are provided which include administering to a patient in need thereof a pharmaceutical composition including gaboxadol or a pharmaceutical salt thereof, in combination with one or more of a CNS stimulant, a eugeroic agent, an antidepressant or a GABA_{B} receptor agonist, wherein the composition provides improvement in one or more symptoms of narcolepsy. In embodiments, methods of treating narcolepsy are provided which include administering to a patient in need thereof a pharmaceutical composition including gaboxadol or a pharmaceutical salt thereof, in combination with one or more of a CNS stimulant, a eugeroic agent, an antidepressant or a GABA_{B} receptor agonist. In embodiments, methods of treating narcolepsy are provided which include administering to a patient in need thereof a pharmaceutical composition including gaboxadol or a pharmaceutical salt thereof, and a pharmaceutical composition including one or more of a CNS stimulant, a eugeroic agent, an antidepressant or a GABA_{B} receptor agonist wherein the compositions provide improvement in one or more symptoms of narcolepsy.

In embodiments, methods of treating narcolepsy are provided which include administering to a patient in need thereof a pharmaceutical composition including gaboxadol or a pharmaceutical salt thereof, in combination with one or more of a CNS stimulant, a eugeroic agent, an antidepressant or a GABA_{B} receptor agonist, wherein the composition provides improvement in one or more symptoms of narcolepsy for more than 6 hours after administration to the patient. In embodiments, methods of treating narcolepsy are provided which include administering to a patient in need thereof a pharmaceutical composition including gaboxadol or a pharmaceutical salt thereof, and a pharmaceutical composition including one or more of a CNS stimulant, a eugeroic agent, an antidepressant or a GABA_{B} receptor agonist, wherein the compositions provide improvement in one or more symptoms of narcolepsy for more than 6 hours after administration to the patient. In embodiments, methods of treating narcolepsy are provided which include administering to a patient in need thereof a pharmaceutical composition including gaboxadol or a pharmaceutically acceptable salt thereof, in combination with one or more of a CNS stimulant, a eugeroic agent, an antidepressant or a GABA_{B} receptor agonist, wherein the composition provides improvement in one or more symptoms of the narcolepsy for more than 8, 10, 12, 14, 16, 18, 20, 22 or 24 hours after administration to the patient. In embodiments, methods of treating narcolepsy are provided which include administering to a patient in need thereof a pharmaceutical composition including gaboxadol or a pharmaceutically acceptable salt thereof, and a pharmaceutical composition including one or more of a CNS stimulant, a eugeroic agent, an antidepressant or a GABA_{B} receptor agonist, wherein the compositions provide improvement in one or more symptoms of the narcolepsy for more than 8, 10, 12, 14, 16, 18, 20, 22 or 24 hours after administration to the patient.

In embodiments, gaboxadol or a pharmaceutically acceptable salt thereof, and one or more of a CNS stimulant, a eugeroic agent, an antidepressant or a GABA_{B} receptor agonist, may be administered in separate dosage forms or combined in one dosage form. In embodiments, gaboxadol or a pharmaceutically acceptable salt thereof may be co-administered simultaneously with one or more of a CNS stimulant, a eugeroic agent, an antidepressant or a GABA_{B} receptor agonist, or at spaced apart intervals. The combination therapies can include administration of the drugs together in the same admixture, or in separate admixtures. In embodiments, the pharmaceutical composition includes two, three, or more drugs.

CNS stimulants include, but are not limited to, amphetamine (dextro and levo amphetamine) (1 mg-60 mg/day), dextroamphetamine (1 mg-60 mg/day), methamphetamine (0.5 mg-30 mg/day), methylphenidate (1 mg-60 mg/day), phentermine (1 mg-50 mg/day), diethylpropion (10 mg-100 mg/day), phendimetrazine (25 mg-250 mg/day), lisdexamfetamine (20 mg-80 mg/day), benzphetamine (25 mg-150 mg/day), atomoxetine (20 mg-100 mg/day), caffeine (10 mg-600 mg/day), and ephedrine (5 mg-150 mg/day).

Eugeroic agents include modafinil (100 mg-300 mg/day) and armodafinil (50 mg-300 mg/day).

Antidepressants include dopamine active anti-depressant agents, dopamine active augmenting agents, serotonin-a norepinephrine reuptake inhibitors (SNRT), norepinephrine reuptake inhibitors, monoamine oxidase inhibitors, tricyclic antidepressants, tetracyclic antidepressants, and selective serotonin re-uptake inhibitors (S SRI). Antidepressants include, but are not limited to, citalopram (10 mg-40 mg/day), escitalopram (5 mg-30 mg/day), paroxetine (10 mg-65 mg/day), fluvoxamine (50 mg-300 mg/day), sertraline (25 mg-200 mg/day), desvenlafaxine (25 mg-400 mg/day), duloxetine (20 mg-120 mg/day), levomilnacipran (10 mg-120 mg/day), milnacipran (10 mg-100 mg/day), venlafaxine (25 mg-375 mg/day), vilazodone (10 mg-50 mg/day) vortioxetine (5 mg-25 mg/day), etoperidone (5 mg-100 mg/day), trazodone (25 mg-400 mg/day), reboxetine (1 mg-10 mg/day), viloxazine (50 mg-600 mg/day), amitriptyline (20 mg-150 mg/day), clomipramine (10 mg-250 mg/day), desipramine (50 mg-300 mg/day), dibenzepin (200 mg-750 mg/day), dosulepin (10 mg-250 mg/day), doxepin (15 mg-150 mg/day), imipramine (50 mg-300 mg/day), lofepramine (15 mg-225 mg/day), melitracen (10 mg-225 mg/day), nitroxazepine (10 mg-225 mg/day), nortriptyline (10 mg-150 mg/day), noxiptiline (5 mg-100 mg/day), pipofezine (5 mg-100 mg/day), protriptyline (5 mg-60 mg/day), trimipramine (25 mg-200 mg/day), amoxapine (25 mg-600 mg/day), maprotiline (50 mg-225 mg/day), mianserin (20 mg-200 mg/day), mirtazapine (5 mg-45 mg/day), setiptiline (1 mg-10 mg/day), isocarboxazid (5 mg-60 mg/day), phenelzine (5 mg-60 mg/day), tranylcypromine (10 mg-60 mg/day), selegiline (1 mg-10 mg/day), moclobemide (100 mg-600 mg/day), pirlindole (25 mg-400 mg/day), toloxatone (100 mg-600 mg/day), amisulpride (200 mg-1200 mg/day), lurasidone (20 mg-160 mg/day), quetiapine (25 mg-750 mg/day), agomelatine (10 mg-50 mg/day), bifemelane (50 mg-150 mg/day), bupropion (50 mg-150 mg/day), ketamine (5 mg-50 mg/day), and tandospirone (10 mg-75 mg/day).

GABA_{B} receptor agonists include, but are not limited to, sodium oxybate (γ-hydroxybutyric acid) (GHB) (0.5 gm-10 gm/day), baclofen (1 mg-80 mg/day) and phenibut (100 mg-2000 mg/day).

Typically, dosages of gaboxadol or a pharmaceutically acceptable salt thereof, and a CNS stimulant, a eugeroic agent, an antidepressant and/or a GABA_{B} receptor agonist, can be administered once daily, twice daily, three times daily, four times daily or more to a patient in need thereof. The methods and compositions described herein may provide reduced dosage amounts which are therapeutically effective, reduced dosing frequency and reduced adverse events and/or increased efficacy. Surprisingly, co-administration of a CNS stimulant, a eugeroic agent, an antidepressant and/or a GABA_{B} receptor agonist, and gaboxadol or a pharmaceutically acceptable salt thereof, may exhibit synergy. For example, the combination of the gaboxadol or a pharmaceutically acceptable salt thereof and one or more of a CNS stimulant, a eugeroic agent, an antidepressant and/or a GABA_{B} receptor agonist provides a therapeutic benefit greater than the additive effect of administering the same dosage of each of the gaboxadol or a pharmaceutically acceptable salt thereof and a CNS stimulant, a eugeroic agent, an antidepressant and/or a GABA_{B} receptor agonist alone. Co-administration of a CNS stimulant, a eugeroic agent, an antidepressant and/or a GABA_{B} receptor agonist, and gaboxadol or a pharmaceutically acceptable salt thereof, can produce a combined effect greater than the sum of their separate effects. Thus, treatment is provided of narcolepsy with a combination of agents that combined, may provide a synergistic effect that enhances efficacy.

In embodiments, gaboxadol or a pharmaceutically acceptable salt thereof in any of the amounts described above is administered to a patient in the evening and a CNS stimulant is administered in the morning. In embodiments, gaboxadol or a pharmaceutically acceptable salt thereof in any of the amounts described above is administered to a patient in the morning and a CNS stimulant is administered in the morning. In embodiments, gaboxadol or a pharmaceutically acceptable salt thereof in any of the amounts described above is administered to a patient in the evening and a eugeroic agent is administered in the morning. In embodiments, gaboxadol or a pharmaceutically acceptable salt thereof in any of the amounts described above is administered to a patient in the morning and a eugeroic agent is administered in the morning. In embodiments, gaboxadol or a pharmaceutically acceptable salt thereof in any of the amounts described above is administered to a patient in the evening and an antidepressant is administered in the morning. In embodiments, gaboxadol or a pharmaceutically acceptable salt thereof in any of the amounts described above is administered to a patient in the morning and an antidepressant is administered in the morning. In embodiments, gaboxadol or a pharmaceutically acceptable salt thereof in any of the amounts described above is administered to a patient in the evening and an antidepressant is administered in the evening. In embodiments, gaboxadol or a pharmaceutically acceptable salt thereof in any of the amounts described above is administered to a patient in the morning and an antidepressant is administered in the evening. In embodiments, gaboxadol or a pharmaceutically acceptable salt thereof in any of the amounts described above is administered to a patient in the evening and a GABA_{B} receptor agonist is administered in the evening. In embodiments, gaboxadol or a pharmaceutically acceptable salt thereof in any of the amounts described above is administered to a patient in the morning and a GABA_{B} receptor agonist is administered in the evening. It should be understood that dosage amounts, frequency and time of administration of aforementioned drugs can be customized to suit individual needs of patients based on their response to administration of the drugs.

Effective treatment of narcolepsy herein whether with gaboxadol or a pharmaceutically acceptable salt thereof alone, or in combination with one or more of a CNS stimulant, a eugeroic agent, an antidepressant and/or a GABA_{B} receptor agonist, may be established by showing reduction in the frequency or severity of symptoms (e.g., more than 10%, 20%, 30% 40% or 50%) after a period of time compared with baseline. For example, after a baseline period of 1 month, the patients may be randomly allocated gaboxadol or a pharmaceutically acceptable salt thereof, either alone or with one or more of a CNS stimulant, a eugeroic agent, an antidepressant and/or a GABA_{B} receptor agonist, or placebo as add-on therapy to standard therapies, during a double-blind period of 2 months. Primary outcome measurements may include the percentage of responders on gaboxadol or a pharmaceutically acceptable salt thereof, either alone or in combination with one or more of a CNS stimulant, a eugeroic agent, an antidepressant and/or a GABA_{B} receptor agonist, and on placebo, defined as having experienced at least a 10% to 50% reduction of symptoms during the second month of the double-blind period compared with baseline.

For example, effective treatment of narcolepsy can include a reduction in the frequency or severity of one or more of excessive daytime sleepiness, abnormal REM sleep, cataplexy, sleep paralysis, hallucinations, automatic behaviors and night-time wakefulness. An effective amount or therapeutically effective amount can be a dosage sufficient to treat, inhibit, or alleviate one or more symptoms of narcolepsy, or to provide a desired pharmacologic and/or physiologic effect, for example, reducing, inhibiting, or reversing one or more of the underlying pathophysiological mechanisms underlying narcolepsy. The precise dosage can vary according to a variety of factors such as subject-dependent variables (e.g., age, immune system health, clinical symptoms etc.).

In embodiments, administration gaboxadol or a pharmaceutically acceptable salt thereof to a patient diagnosed with narcolepsy is effective to beneficially delay onset of REM sleep in the patient. As mentioned above, people with narcolepsy frequently abnormally enter REM sleep within 15 minutes of falling asleep. Surprisingly, it has been found that administration of 0.5 mg to 25 mg of gaboxadol or a pharmaceutically acceptable salt thereof to a narcoleptic patient can delay onset of REM sleep to 30 minutes or more after falling asleep. Without wishing to be bound by any theory, symptoms associated with narcolepsy such as cataplexy, sleep paralysis, hallucinations, and automatic behaviors closely mimic the natural physiologic response that occurs during REM sleep. By inducing a more normal REM sleep architecture in narcoleptic patients, symptoms associated with narcolepsy are reduced or alleviated. Accordingly, a method of treating narcolepsy is provided that includes administering gaboxadol or a pharmaceutically acceptable salt thereof to a patient in need thereof in an amount effective to delay onset of REM sleep in the patient after falling asleep. In embodiments, the delay of onset of REM sleep is about 30 minutes or longer. In embodiments, the delay of onset of REM sleep is about 45 minutes or longer. In embodiments, the delay of onset of REM sleep is about 60 minutes or longer. In embodiments, the delay of onset of REM sleep is about 75 minutes or longer. In embodiments, the amount of gaboxadol effective to beneficially delay onset of REM sleep may range from 5 mg to 30 mg. In embodiments, the amount of gaboxadol effective to beneficially delay onset of REM sleep may range from 5 mg to 25 mg. In embodiments, the amount of gaboxadol effective to beneficially delay onset of REM sleep may range from 5 mg to 20 mg. In embodiments, the amount of gaboxadol effective to beneficially delay onset of REM sleep may range from 5 mg to 15 mg. In embodiments, the amount of gaboxadol effective to beneficially delay onset of REM sleep may range from 5 mg to 10 mg.

As mentioned previously pharmaceutical compositions herein may be conventional or modified, i.e., provided with conventional release profiles or modified release profiles. Conventional (or unmodified) release oral dosage forms such as tablets or capsules typically release medications into the stomach or intestines as the tablet or capsule shell dissolves. The pattern of drug release from modified release (MR) dosage forms is deliberately changed from that of a conventional dosage form to achieve a desired therapeutic objective and/or better patient compliance. Types of MR drug products include orally disintegrating dosage forms (ODDFs) which provide immediate release, extended release dosage forms, delayed release dosage forms (e.g., enteric coated), and pulsatile release dosage forms. In embodiments, pharmaceutical compositions with different drug release profiles may be combined to create a two phase or three-phase release profile. For example, pharmaceutical compositions may be provided with an immediate release and an extended release profile. In embodiments, pharmaceutical compositions may be provided with an extended release and delayed release profile. Such composition may be provided as pulsatile formulations, multilayer tablets, or capsules containing tablets, beads, granules, etc. Compositions may be prepared using a pharmaceutically acceptable “carrier” composed of materials that are considered safe and effective. The “carrier” includes all components present in the pharmaceutical formulation other than the active ingredient or ingredients. The term “carrier” includes, but is not limited to, diluents, binders, lubricants, disintegrants, fillers, and coating compositions.

An ODDF is a solid dosage form containing a medicinal substance or active ingredient which disintegrates rapidly, usually within a matter of seconds when placed upon the tongue. The disintegration time for ODDFs generally range from one or two seconds to about a minute. ODDFs are designed to disintegrate or dissolve rapidly on contact with saliva. This mode of administration can be beneficial to people who may have problems swallowing tablets whether it be from physical infirmity or psychiatric in nature. In embodiments, when administered to an oral cavity, an ODDF herein disintegrates in less than one minute, less than 55 seconds, less than 50 seconds, less than 45 seconds, less than 40 seconds, less than 35 seconds, less than 30 seconds, less than 25 seconds, less than 20 seconds, less than 15 seconds, less than 10 seconds, or less than 5 seconds.

An orally disintegrating tablet (ODT) is a solid dosage form containing a medicinal substance or active ingredient which disintegrates rapidly, usually within a matter of seconds when placed upon the tongue. The disintegration time for ODTs generally ranges from several seconds to about a minute. ODTs are designed to disintegrate or dissolve rapidly on contact with saliva, thus eliminating the need to chew the tablet, swallow the intact tablet, or take the tablet with liquids. In embodiments, an ODT herein disintegrates in less than one minute, less than 55 seconds, less than 50 seconds, less than 45 seconds, less than 40 seconds, less than 35 seconds, less than 30 seconds, less than 25 seconds, less than 20 seconds, less than 15 seconds, less than 10 seconds, or less than 5 seconds, based upon, e.g., the United States Pharmacopeia (USP) disintegration test method set forth at section 701, Revision Bulletin Official Aug. 1, 2008.

Other ODDFs which may be used herein include rapidly dissolving films which are thin oral strips that release medication such as gaboxadol or a pharmaceutically acceptable salt thereof quickly after administration to the oral cavity. The film is placed on a patient's tongue or any other mucosal surface and is instantly wet by saliva whereupon the film rapidly hydrates and dissolves to release the medication. See. e.g., Chaturvedi et al., Curr Drug Deliv. 2011 July; 8(4):373-80. Fastcaps are a rapidly disintegrating drug delivery system based on gelatin capsules. Freeze dried (lyophilized) wafers are rapidly disintegrating, thin matrixes that contain a medicinal agent. The wafer or film disintegrates rapidly in the oral cavity and releases drug which dissolves or disperses in the saliva. See, e.g., Boateng et al., Int J Pharm. 2010 Apr. 15; 389(1-2):24-31. Those skilled in the art are familiar with various techniques utilized to manufacture ODDFs such as freeze drying, spray drying, phase transition processing, melt granulation, sublimation, mass extrusion, cotton candy processing, direct compression, etc.

When administered, ODDFs containing gaboxadol or a pharmaceutically acceptable salt thereof, either alone or in combination with one or more additional drugs discussed herein, e.g., a CNS stimulant, a eugeroic agent, an antidepressant and a GABA_{B} receptor agonist (collectively referred to herein as “drug”, “drugs”, “active agent”, or “active agents”), disintegrate rapidly to release the drug(s), which dissolves or disperses in the saliva. The drug may be absorbed in the oral cavity, e.g., sublingually, buccally, from the pharynx and esophagus or from other sections of gastrointestinal tract as the saliva travels down. In such cases, bioavailability can be significantly greater than that observed from conventional tablet dosage forms which travel to the stomach or intestines where drug can be released.

In embodiments, pharmaceutical compositions having modified release profiles provide pharmacokinetic properties which result in both rapid onset and sustained duration of action. Such pharmaceutical compositions can include an immediate release aspect and an extended release aspect. Immediate release aspects are discussed above in connection with ODDFs. Extended release dosage forms (ERDFs) have extended release profiles and are those that allow a reduction in dosing frequency as compared to that presented by a conventional dosage form, e.g., a solution or unmodified release dosage form. ERDFs provide a sustained duration of action of a drug. In embodiments, modified release dosage forms herein can be ERDFs that do not have an ODDF aspect. In embodiments, modified release dosage forms herein incorporate an ODDF aspect to provide immediate release of a loading dose and then an ERDF aspect that provides prolonged delivery to maintain drug levels in the blood within a desired therapeutic range for a desirable period of time in excess of the activity resulting from a single dose of the drug. In embodiments, the ODDF aspect releases the drug immediately and the ERDF aspect thereafter provides continuous release of drug for sustained action.

In embodiments, an ODDF can be applied as a coating or band over an ERDF, or as a layer adjacent to an ERDF, to allow direct exposure of the ODDF to the oral cavity and consequent disintegration of the ODDF. In embodiments, the ODDF and the ERDF can be mixed in a chewable resin, e.g., gum. Those skilled in the art are familiar with techniques for applying coatings, bands and layers to fabricate pharmaceutical dosage forms.

Suitable formulations which provide extended release profiles are well-known in the art. For example, coated slow release beads or granules (“beads” and “granules” are used interchangeably herein) in which, e.g., gaboxadol or a pharmaceutically acceptable salt thereof, alone or in combination with one or more drugs, is applied to beads, e.g., confectioners nonpareil beads, and then coated with conventional release retarding materials such as waxes, enteric coatings and the like. In embodiments, some beads incorporate one drug while other beads incorporate a different drug. In embodiments, beads can be formed in which one or more drugs are mixed with a material to provide a mass from which the drug leaches out. In embodiments, the beads may be engineered to provide different rates of release by varying characteristics of the coating or mass, e.g., thickness, porosity, using different materials, etc. Beads having different rates of release may be combined into a single dosage form to provide variable or continuous release. The beads can be contained in capsules or compressed into tablets. In embodiments, the ODDF is applied as a coating, a layer or a band to a capsule or tablet. In embodiments, slow release cores which are incorporated into tablets or capsules can also provide extended release profiles. For example, one or more drugs can be mixed in a substance or a mixture of substances non-absorbable from the gastrointestinal tract but capable of slow dissolution or loss of drug by leaching, and an outer drug containing ODDF layer which is applied to the core by, e.g., compression or spraying. In embodiments, extended release profiles may be provided by multiple layer tablets, each layer having different release properties. Multilayer tableting machines allow incorporation into one tablet of two or more separate layers which may be made to release one or more drugs at different rates. For example, one or more outer layers may be an ODDF, and each other layer an ERDF that exhibits different release rates. In embodiments, one or more drugs are incorporated into porous inert carriers that provide extended release profiles. In embodiments, the porous inert carriers incorporate channels or passages from which the drug diffuses into surrounding fluids. In embodiments, one or more drugs are incorporated into an ion-exchange resin to provide an extended release profile. Prolonged action results from a predetermined rate of release of the drug from the resin when the drug-resin complex contacts gastrointestinal fluids and the ionic constituents dissolved therein. In embodiments, membranes are utilized to control rate of release from drug containing reservoirs. In embodiments, liquid preparations may also be utilized to provide an extended release profile. For example, a liquid preparation consisting of solid particles dispersed throughout a liquid phase in which the particles are not soluble. The suspension is formulated to allow at least a reduction in dosing frequency as compared to that drug presented as a conventional dosage form (e.g., as a solution or a prompt drug-releasing, conventional solid dosage form). For example, a suspension of ion-exchange resin constituents or microbeads.

In embodiments, absorbable or non-absorbable polymers may be utilized to form ERDFs. Various ERDFs including those discussed above and others that can be utilizable herein are known to those with skill in the art. See, e.g., Fu and Kao, Expert Opin Drug Deliv. 2010 April; 7(4): 429-444.

In embodiments, modified dosage forms herein incorporate delayed release dosage forms having delayed release profiles. Delayed release dosage forms can include delayed release tablets or delayed release capsules. A delayed release tablet is a solid dosage form which releases a drug (or drugs) such as gaboxadol or a pharmaceutically acceptable salt thereof at a time other than promptly after administration. A delayed release capsule is a solid dosage form in which the drug is enclosed within either a hard or soft soluble container made from a suitable form of gelatin, and which releases a drug (or drugs) at a time other than promptly after administration. For example, with respect to tablets or capsules, enteric-coated articles are examples of delayed release dosage forms. In embodiments, a delayed release tablet is a solid dosage form containing a conglomerate of medicinal particles that releases a drug (or drugs) at a time other than promptly after administration. In embodiments, the conglomerate of medicinal particles are covered with a coating which delays release of the drug. In embodiments, a delayed release capsule is a solid dosage form containing a conglomerate of medicinal particles that releases a drug (or drugs) at a time other than promptly after administration. In embodiments, the conglomerate of medicinal particles are covered with a coating which delays release of the drug.

In embodiments, ODDFs with a delayed release formulation aspect are provided that are solid dosage forms containing medicinal substances which disintegrate rapidly, usually within a matter of seconds, when placed upon the tongue, but which also releases a drug (or drugs) at a time other than promptly after administration. Accordingly, in embodiments, modified release dosage forms herein incorporate an ODDF aspect to provide immediate release of a loading dose and then an a delayed release formulation aspect that provides a period in which there is no drug delivery followed by a period of drug delivery to provide drug levels in the blood within a desired therapeutic range for a desirable period of time in excess of the activity resulting from a single dose of the drug. In embodiments, the ODDF aspect releases drug immediately and then, after a period of delay, a delayed release formulation aspect thereafter provides a single release of drug to provide an additional period of activity. In embodiments, the ODDF aspect releases the drug immediately and then, after a period of delay, a delayed release formulation aspect thereafter provides a continuous release of drug for sustained action. In embodiments, different drugs are released together or at different times.

Delayed release dosage forms are known to those skilled in the art. For example, coated delayed release beads or granules (“beads” and “granules” are used interchangeably herein) in which, e.g., gaboxadol or a pharmaceutically acceptable salt thereof and/or other drug is applied to beads, e.g., confectioners nonpareil beads, and then coated with conventional release delaying materials such as waxes, enteric coatings and the like. In embodiments, beads can be formed in which drug is mixed with a material to provide a mass from which the drug leaches out. In embodiments, the beads may be engineered to provide different rates of release by varying characteristics of the coating or mass, e.g., thickness, porosity, using different materials, etc. In embodiments, enteric coated granules of drug can be contained in an enterically coated capsule or tablet which releases the granules in the small intestine. In embodiments, the granules have a coating which remains intact until the coated granules reach at least the ileum and thereafter provide a delayed release of the drug in the colon. Suitable enteric coating materials are well known in the art, e.g., Eudragit® coatings such methacrylic acid and methyl methacrylate polymers and others. The granules can be contained in capsules or compressed into tablets. In embodiments, the ODDF is applied as a coating, a layer or a band to the capsule or tablet. In embodiments, delayed release cores which are incorporated into tablets or capsules can also provide delayed release profiles. For example, gaboxadol or a pharmaceutically acceptable salt thereof can be mixed in a substance or a mixture of substances non-absorbable from the gastrointestinal tract but capable of slow dissolution or loss of drug by leaching, and an outer ODDF layer which is applied to the core by, e.g., compression or spraying. In embodiments, delayed release profiles may be provided by multiple layer tablets, each layer having different release properties. Multilayer tableting machines allow incorporation into one tablet of two or more separate layers which may be made to release drug at different rates after a period of delay. For example, one or more outer layers may be an ODDF, and each other layer a delayed release dosage form that exhibits different release rates. In embodiments, drug is incorporated into porous inert carriers that provide delayed release profiles. In embodiments, the porous inert carriers incorporate channels or passages from which the drug diffuses into surrounding fluids. In embodiments, drug is incorporated into an ion-exchange resin to provide a delayed release profile. Delayed action may result from a predetermined rate of release of the drug from the resin when the drug-resin complex contacts gastrointestinal fluids and the ionic constituents dissolved therein. In embodiments, membranes are utilized to control rate of release from drug containing reservoirs. In embodiments, liquid preparations may also be utilized to provide a delayed release profile. For example, a liquid preparation consisting of solid particles dispersed throughout a liquid phase in which the particles are not soluble. The suspension is formulated to allow at least a reduction in dosing frequency as compared to that drug presented as a conventional dosage form (e.g., as a solution or a prompt drug-releasing, conventional solid dosage form). For example, a suspension of ion-exchange resin constituents or microbeads.

In embodiments, modified release pharmaceutical compositions herein include pulsatile release dosage formulations (PRDFs). Pulsatile drug release involves rapid release of defined or discrete amounts of a drug (or drugs) such as gaboxadol or a pharmaceutically acceptable salt thereof after a lag time following an initial release of drug. In embodiments, PRDFs can provide a single pulse. In embodiments, PRDFs can provide multiple pulses over time. Various PRDFs are known to those with skill in the art.

In embodiments, a PRDF can be a capsule. In embodiments, release after a lag time is provided by a system that uses osmotic pressure to cause release of a plug. In this system, gaboxadol or a pharmaceutically acceptable salt thereof is contained in an insoluble capsule shell sealed by an osmotically responsive plug, e.g., a hydrogel, which is pushed away by swelling or erosion. When the seal is broken the drug is released as a pulse from the capsule body. Contact with gastrointestinal fluid or dissolution medium causes the plug to swell, either pushing itself out of the capsule or causing the capsule to rupture after the lag-time. Position & dimensions of the plug can control lag-time. For rapid release of drug effervescent or disintegrating agents may be added. Effervescent materials can cause an increase in pressure thus aiding or causing expulsion of the plug. Examples of suitable plug material may be swellable materials coated with permeable polymer (polymethacrylates), erodible compressed polymer (HPMC, polyvinyl alcohol), congealed melted polymer (glyceryl monooleate), and enzymatically controlled erodible polymers such as pectin. In embodiments, an insoluble capsule contains multiple drug compartments separated by osmotically activated plugs. When a first plug is exposed to the environmental fluids, the first compartment opens, drug is released and the adjacent plug is exposed. The process continues until no sealed compartment are left. Lag time between pulses can be further controlled by varying the thickness of the plug and the properties of the materials from which the plug is made. More hygroscopic materials will absorb fluid faster and will swell faster. In embodiments, a membrane may be substituted for the plug. If effervescent materials are included in one or more compartments, fluids pass through the membrane by osmosis and the effervescent action and pressure increase causes the membrane to rupture, thereby releasing the drug. In embodiments, the membrane(s) are erodible and dissolve to release the contents of the compartment(s). Varying the thickness, porosity and properties of materials of the membrane can allow further control of lag time between pulses. In embodiments, a PRDF can be a tablet. In embodiments, single pulse tablets involve a core containing gaboxadol or a pharmaceutically acceptable salt thereof surrounded by one or more layers of swellable, rupturable coatings. In embodiments, a rupturable coating surrounds a swellable layer. As the swellable layer expands, it causes the rupturable coating to rupture, thereby releasing the drug from the core. Swellable materials such as hydrogels are well known. In embodiments, an inner swelling layer can contain a superdisintegrant, e.g., croscarmellose sodium, and an outer rupturable layer can be made of a polymeric porous materials such as polyethylene oxides, ethylcellulose and the like. Porous film coats of sucrose may also be suitable. In embodiments, multiple pulse tablets incorporate multiple layers surrounding a core. As a first outermost layer erodes and releases the drug contained within the layer, an underlying layer is exposed, thus releasing drug after a predetermined lag time. The process repeats until the innermost core is exposed.

In embodiments, PRDFs can incorporate ODDFs that are solid dosage forms containing medicinal substances which disintegrate rapidly, usually within a matter of seconds, when placed upon the tongue, but which also releases a drug (or drugs) in pulsatile fashion. Accordingly, in embodiments, modified release dosage forms herein can incorporate an ODDF aspect to provide immediate release of a loading dose and a PRDF aspect that provides a period in which there is no drug delivery (lag time) followed by pulsatile drug delivery to provide drug levels in the blood within a desired therapeutic range for a desirable period of time in excess of the activity resulting from a single dose of the drug. In embodiments, the ODDF aspect releases the drug immediately and then, after a period of delay, the PRDF aspect thereafter provides a single pulse release of drug to provide an additional period of activity. In embodiments, the ODDF aspect releases the drug immediately and then, after a period of delay, the PRFD aspect thereafter provides multiple pulsatile release of drug for prolonged therapeutic effect.

In embodiments, an ODDF is applied as a coating or band over a PRDF, or as a layer adjacent to a PRDF, to allow direct exposure of the ODDF to the oral cavity and consequent disintegration of the ODDF. In embodiments, the ODDF and a PRDF can be mixed in a chewable resin, e.g., gum. Those skilled in the art are familiar with techniques for applying coatings, bands and layers to fabricate pharmaceutical dosage forms.

In embodiments, the pharmaceutical compositions, including those that are modified release formulations, can include 0.1 mg to 75 mg, 0.1 mg to 70 mg, 0.1 mg to 65 mg, 0.1 mg to 55 mg, 0.1 mg to 50 mg, 0.1 mg to 45 mg, 0.1 mg to 40 mg, 0.1 mg to 35 mg, 0.1 mg to 30 mg, 0.1 mg to 25 mg, 0.1 mg to 20 mg, 0.1 mg to 15 mg, 0.1 mg to 10 mg, 0.5 mg to 75 mg, 0.5 mg to 70 mg, 0.5 mg to 65 mg, 0.5 mg to 55 mg, 0.5 mg to 50 mg, 0.5 mg to 45 mg, 0.5 mg to 40 mg, 0.5 mg to 35 mg, 0.5 mg to 30 mg, 0.5 mg to 25 mg, 0.5 mg to 20 mg, 0.5 to 15 mg, 0.5 to 10 mg, 1 mg to 75 mg, 1 mg to 70 mg, 1 mg to 65 mg, 1 mg to 55 mg, 1 mg to 50 mg, 1 mg to 45 mg, 1 mg to 40 mg, 1 mg to 35 mg, 1 mg to 30 mg, 1 mg to 25 mg, 1 mg to 20 mg, 1 mg to 15 mg, 1 mg to 10 mg, 1.5 mg to 75 mg, 1.5 mg to 70 mg, 1.5 mg to 65 mg, 1.5 mg to 55 mg, 1.5 mg to 50 mg, 1.5 mg to 45 mg, 1.5 mg to 40 mg, 1.5 mg to 35 mg, 1.5 mg to 30 mg, 1.5 mg to 25 mg, 1.5 mg to 20 mg, 1.5 mg to 15 mg, 1.5 mg to 10 mg, 2 mg to 75 mg, 2 mg to 70 mg, 2 mg to 65 mg, 2 mg to 55 mg, 2 mg to 50 mg, 2 mg to 45 mg, 2 mg to 40 mg, 2 mg to 35 mg, 2 mg to 30 mg, 2 mg to 25 mg, 2 mg to 20 mg, 2 mg to 15 mg, 2 mg to 10 mg, 2.5 mg to 75 mg, 2.5 mg to 70 mg, 2.5 mg to 65 mg, 2.5 mg to 55 mg, 2.5 mg to 50 mg, 2.5 mg to 45 mg, 2.5 mg to 40 mg, 2.5 mg to 35 mg, 2.5 mg to 30 mg, 2.5 mg to 25 mg, 2.5 mg to 20 mg, 2.5 mg to 15 mg, 2.5 mg to 10 mg, 3 mg to 75 mg, 3 mg to 70 mg, 3 mg to 65 mg, 3 mg to 55 mg, 3 mg to 50 mg, 3 mg to 45 mg, 3 mg to 40 mg, 3 mg to 35 mg, 3 mg to 30 mg, 3 mg to 25 mg, 3 mg to 20 mg, 3 mg to 15 mg, 3 mg to 10 mg, 3.5 mg to 75 mg, 3.5 mg to 70 mg, 3.5 mg to 65 mg, 3.5 mg to 55 mg, 3.5 mg to 50 mg, 3.5 mg to 45 mg, 3.5 mg to 40 mg, 3.5 mg to 35 mg, 3.5 mg to 30 mg, 3.5 mg to 25 mg, 3.5 mg to 20 mg, 3.5 mg to 15 mg, 3.5 mg to 10 mg, 4 mg to 75 mg, 4 mg to 70 mg, 4 mg to 65 mg, 4 mg to 55 mg, 4 mg to 50 mg, 4 mg to 45 mg, 4 mg to 40 mg, 4 mg to 35 mg, 4 mg to 30 mg, 4 mg to 25 mg, 4 mg to 20 mg, 4 mg to 15 mg, 4 mg to 10 mg, 4.5 mg to 75 mg, 4.5 mg to 70 mg, 4.5 mg to 65 mg, 4.5 mg to 55 mg, 4.5 mg to 50 mg, 4.5 mg to 45 mg, 4.5 mg to 40 mg, 4.5 mg to 35 mg, 4.5 mg to 30 mg, 4.5 mg to 25 mg, 4.5 mg to 20 mg, 4.5 mg to 15 mg, 4.5 mg to 10 mg, 5 mg to 75 mg, 5 mg to 70 mg, 5 mg to 65 mg, 5 mg to 55 mg, 5 mg to 50 mg, 5 mg to 45 mg, 5 mg to 40 mg, 5 mg to 35 mg, 5 mg to 30 mg, 5 mg to 25 mg, 5 mg to 20 mg, 5 mg to 15 mg, or 5 mg to 10 mg, gaboxadol or a pharmaceutically acceptable salt thereof.

In embodiments, pharmaceutical compositions include 5 mg to 20 mg, 5 mg to 10 mg, 4 mg to 6 mg, 6 mg to 8 mg, 8 mg to 10 mg, 10 mg to 12 mg, 12 mg to 14 mg, 14 mg to 16 mg, 16 mg to 18 mg, or 18 mg to 20 mg gaboxadol or a pharmaceutically acceptable salt thereof.

In embodiments, pharmaceutical compositions include 0.1 mg, 0.25 mg, 0.5 mg, 1 mg, 2.5 mg, 3 mg, 4 mg, 5 mg, 7 mg, 7.5 mg, 10 mg, 12.5 mg, 15 mg, 17.5 mg, or 20 mg gaboxadol or a pharmaceutically acceptable salt thereof or amounts that are multiples of such doses. In embodiments, pharmaceutical compositions include 2.5 mg, 5 mg, 7.5 mg, 10 mg, 15 mg, or 20 mg gaboxadol or a pharmaceutically acceptable salt thereof.

In embodiments, ODDFs include 0.05 mg, 0.1 mg, 0.25 mg, 0.5 mg, 0.75 mg, 1 mg, 1.25 mg, 1.5 mg, 1.75 mg, 2 mg, 2.5 mg, 3 mg, 3.5 mg, 4 mg, 4.5 mg, 5 mg, 7 mg, 7.5 mg, 10 mg, 12.5 mg, 15 mg, 17.5 mg, or 20 mg gaboxadol or a pharmaceutically acceptable salt thereof or amounts that are multiples of such doses.

In embodiments, ERDFs include from about 1 mg to about 100 mg gaboxadol or a pharmaceutically acceptable salt thereof. In embodiments, ERDFs include 5 mg, 6 mg, 7 mg, 8 mg, 9 mg, 10 mg, 11 mg, 12 mg, 13 mg, 14 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, or 100 mg gaboxadol or a pharmaceutically acceptable salt thereof.

In embodiments, delayed release dosage forms include from about 0.05 mg to about 100 mg gaboxadol or a pharmaceutically acceptable salt thereof. In embodiments, delayed release dosage forms include 0.05 mg, 0.1 mg, 0.25 mg, 0.5 mg, 0.75 mg, 1 mg, 1.25 mg, 1.5 mg, 1.75 mg, 2 mg, 2.5 mg, 3 mg, 3.5 mg, 4 mg, 4.5 mg, 5 mg, 6 mg, 7 mg, 8 mg, 9 mg, 10 mg, 11 mg, 12 mg, 13 mg, 14 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, or 100 mg gaboxadol or a pharmaceutically acceptable salt thereof.

In embodiments, PRDFs include one or more pulse providing domains having from about 0.05 mg to about 100 mg gaboxadol or a pharmaceutically acceptable salt thereof. In embodiments, PRDFs include 0.05 mg, 0.1 mg, 0.25 mg, 0.5 mg, 0.75 mg, 1 mg, 1.25 mg, 1.5 mg, 1.75 mg, 2 mg, 2.5 mg, 3 mg, 3.5 mg, 4 mg, 4.5 mg, 5 mg, 6 mg, 7 mg, 8 mg, 9 mg, 10 mg, 11 mg, 12 mg, 13 mg, 14 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, or 100 mg gaboxadol or a pharmaceutically acceptable salt thereof.

In embodiments, the respective daily amounts of a CNS stimulant, a eugeroic agent, an antidepressant or a GABA_{B} receptor agonist as discussed above can be administered in combination with gaboxadol or a pharmaceutically acceptable salt thereof in the amounts and dosage forms discussed above. The respective daily amounts may be administered all at once or in divided doses. It should be understood that the ranges of daily dosages discussed above include every integer and tenth of an integer between the low amount and high amount as if fully set forth herein.

Unless defined otherwise, all technical and scientific terms used herein have the same meanings as commonly understood by one of skill in the art to which the disclosure herein belongs.

The term “about” or “approximately” as used herein means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, up to 10%, up to 5%, and/or up to 1% of a given value. Alternatively, particularly with respect to biological systems or processes, the term can mean within an order of magnitude, preferably within 5-fold, and more preferably within 2-fold, of a value.

“Improvement” refers to the treatment of symptoms or conditions associated with narcolepsy, measured relative to at least one symptom or condition of narcolepsy.

“Improvement in next day functioning” or “wherein there is improvement in next day functioning” refers to improvement after waking from an overnight sleep period wherein the beneficial effect of administration of gaboxadol or a pharmaceutically acceptable salt thereof, alone or in combination with one or more of a CNS stimulant, a eugeroic agent, an antidepressant or a GABA_{B} receptor agonist, applies to at least one symptom or condition associated with narcolepsy and is discernable, either subjectively by a patient or objectively by an observer, for a period of time, e.g., 2 hours, 3 hours, 4 hours, 5 hours, 6 hours, 12 hours, 24 hours, etc. after waking.

“Treat”, “Treating” or “treatment” refers to alleviating or delaying the appearance of clinical symptoms of a disease or condition in a subject that may be afflicted with or predisposed to the disease or condition, but does not yet experience or display clinical or subclinical symptoms of the disease or condition. In certain embodiments, “treat”, “treating” or “treatment” may refer to preventing the appearance of clinical symptoms of a disease or condition in a subject that may be afflicted with or predisposed to the disease or condition, but does not yet experience or display clinical or subclinical symptoms of the disease or condition. “Treat”, “treating” or “treatment” also refers to inhibiting the disease or condition, e.g., arresting or reducing its development or at least one clinical or subclinical symptom thereof. “Treat”, “treating” or “treatment” further refers to relieving the disease or condition, e.g., causing regression of the disease or condition or at least one of its clinical or subclinical symptoms. The term “treat” “treating” or “treatment” may mean to relieve or alleviate the intensity and/or duration of a manifestation of a disease experienced by a subject in response to a given stimulus (e.g., pressure, tissue injury, cold temperature, etc.). The benefit to a subject to be treated may be statistically significant, mathematically significant, or at least perceptible to the subject and/or the physician.

“Patient in need thereof” may include individuals that have been diagnosed with narcolepsy. “Patient” and “subject” are used interchangeably herein.

“Effective amount” or “therapeutically effective amount” means a dosage sufficient to alleviate one or more symptoms of a disorder, disease, or condition being treated, or to otherwise provide a desired pharmacological and/or physiologic effect. The “effective amount” or “therapeutically effective amount” can vary depending on the compound, the disease and its severity and the age, weight, physical condition and responsiveness of the subject to be treated. In embodiments, a therapeutically effective amount of active agent(s) is an amount effective to treat narcolepsy. The effective amount of the drug for pharmacological action, and therefore the dosage strength may depend on progression of the disease itself “Effective amount” or “therapeutically effective amount” may be used interchangeably herein.

“Pharmaceutically acceptable” refers to molecular entities and compositions that are “generally regarded as safe”, e.g., they are biologically or pharmacologically compatible for in vivo use in animals or humans, that are physiologically tolerable and do not typically produce an allergic or similar untoward reaction, when administered to a human. In embodiments, this term refers to molecular entities and compositions approved by a regulatory agency of the federal or a state government, as the GRAS list under section 204(s) and 409 of the Federal Food, Drug and Cosmetic Act, that is subject to premarket review and approval by the FDA or similar lists, the U.S. Pharmacopeia or another generally recognized pharmacopeia for use in animals, and more particularly in humans.

The term “pharmaceutically acceptable salt”, as used herein, refers to derivatives of the compounds defined herein, wherein the parent compound is modified by making acid or base salts thereof. Example of pharmaceutically acceptable salts include but are not limited to mineral or organic acid salts of basic residues such as amines; and alkali or organic salts of acidic residues such as carboxylic acids. The pharmaceutically acceptable salts include the conventional non-toxic salts or the quaternary ammonium salts of the parent compound formed, for example, from non-toxic inorganic or organic acids. Such conventional non-toxic salts include those derived from inorganic acids such as hydrochloric, hydrobromic, sulfuric, sulfamic, phosphoric, and nitric acids; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, tolunesulfonic, naphthalenesulfonic, methanesulfonic, ethane disulfonic, oxalic, and isethionic salts. The pharmaceutically acceptable salts can be synthesized from the parent compound, which contains a basic or acidic moiety, by conventional chemical methods.

“Co-administered with”, “in combination with”, “administered in combination with”, “a combination of or” administered along with” may be used interchangeably and mean that two or more agents are administered in the course of therapy. The agents may be administered together at the same time or separately in spaced apart intervals. The agents may be administered in a single dosage form or in separate dosage forms.

As used herein, “sustained release” or “extended release” means that the release of the therapeutically active agent (drug) occurs over an extended period of time leading to lower peak plasma concentrations and/or is directed to a prolonged Tₘₐₓ as compared to “conventional release” or “immediate release.” For example, extended release compositions may have a mean Tₘₐₓ of about 5 or more hours. 3

The term “dissolution requirement” means the dissolution rate of dosage forms, including bead-based dosage forms, obtained when tested using the equipment and procedure specified in the USP XXV and conducted pursuant to the individual Official Monographs of USP XXV for the particular therapeutically active agent(s).

“PK” refers to the pharmacokinetic profile. Cₘₐₓ is defined as the highest plasma drug concentration estimated during an experiment (ng/ml). Tₘₐₓ is defined as the time when Cₘₐₓ is estimated (min). AUC_{0-∞} is the total area under the plasma drug concentration-time curve, from drug administration until the drug is eliminated (ng·hr/ml or μg·hr/ml). The area under the curve is governed by clearance. Clearance is defined as the volume of blood or plasma that is totally cleared of its content of drug per unit time (ml/min).

“Prodrug” refers to a pharmacological substance (drug) that is administered to a subject in an inactive (or significantly less active) form. Once administered, the prodrug is metabolized in the body (in vivo) into a compound having the desired pharmacological activity.

“Analog” and “Derivative” may be used interchangeably and refer to a compound that possesses the same core as the parent compound, but may differ from the parent compound in bond order, the absence or presence of one or more atoms and/or groups of atoms, and combinations thereof. The derivative can differ from the parent compound, for example, in one or more substituents present on the core, which may include one or more atoms, functional groups, or substructures. In general, a derivative can be imagined to be formed, at least theoretically, from the parent compound via chemical and/or physical processes.

### EXAMPLES

The Examples provided herein are included solely for augmenting the disclosure herein and should not be considered to be limiting in any respect.

### Example 1

The following Example provides the plasma concentration profiles and dose proportionality of gaboxadol monohydrate following single oral doses ranging from 2.5 to 20 mg. The absolute bioavailability of gaboxadol monohydrate capsules ranging from 2.5 to 20 mg is also assessed.

This study was composed of separate groups of 10 healthy adult subjects (at least 4 of each gender) who participated in a 6-period, double-blind, randomized, crossover study designed to access the dose proportionality and absolute bioavailabilty of 5 single oral doses of gaboxadol across the dose range of 2.5 to 20 mg. The order in which the subjects received the 5 single oral doses of gaboxadol (2.5; 5; 10; 15; and 20 mg) was randomized within Treatment Periods 1 through 5. Each subject was expected to complete all 6 treatment periods and there was a washout of at least 4 days between each treatment period.

Each oral dosing within Treatment Periods consisted of 2 capsules of test drug taken simultaneously at each scheduled dosing. The treatment designations for the orally administered study drugs were as follows: Treatment A—one 2.5 mg gaboxadol capsule and 1 matching placebo capsule; Treatment B—one 5 mg gaboxadol capsule and 1 matching placebo capsule; Treatment C—one 10 mg gaboxadol capsule and 1 matching placebo capsule; Treatment D—one 15 mg gaboxadol capsule and 1 matching placebo capsule; and Treatment E—20 mg gaboxadol (two 10 mg gaboxadol capsules). Subjects received their study drug after an overnight fast with 240 mL of water in the morning about 8:00 AM. Water was permitted ad libitum except within 1 hour prior to and after study drug administration. No food was allowed for 4 hours post dose.

For each subject in each treatment, plasma and urine samples were collected over 16 hours post-dosing for the determination of pharmacokinetic parameters (e.g., AUC, Cₘₐₓ, Tₘₐₓ, apparent t½, cumulative urinary excretion, renal clearance, clearance, and steady-state volume of distribution, as appropriate). AUC and Cₘₐₓ for gaboxadol were potency adjusted to facilitate comparison of pharmacokinetic data across studies. Table 1 provides the individual potency-adjusted pharmacokinetic parameters of gaboxadol following single oral doses (2.5, 5, 10, 15, and 20 mg).

TABLE 1
Table 1. Pharmacokinetic parameters for gaboxadol following oral and IV administration
Pharmacokinetic parameters for gaboxadol following oral and IV administration

| | Geometric Mean (N = 10) | | | | | | |
|---|---|---|---|---|---|---|---|
| | | | 10 mg | 10 mg | | | |
| Parameter | 2.5 mg | 5 mg | Oral | I.V. | 15 mg | 20 mg | Slope (90% CI) ^{††} |
| AUC_{0-∞} (ng · hr/mL) | 90 | 171 | 346 | 380 | 539 | 669 | 0.98 (0.95, 1.01) |
| Cₘₐₓ (ng/mL)^{†} | 61 | 110 | 232 | 212 | 382 | 393 | 0.95 (0.88, 1.02) |
| Tₘₐₓ (hr)^{‡} | 0.5 | 0.6 | 0.5 | — | 0.5 | 0.6 | |
| Apparent t_{1/2} (hr)^{§} | 1.5 | 1.5 | 1.6 | 1.5 | 1.5 | 1.6 | |
| CL/F (mL/min)^{ϑ} | 461 | 488 | 476 | 438 | 469 | 499 | |
| Fₑ (%) | 43 | 45 | 53 | 53 | 50 | 53 | |
| CL_{R} (mL/min) | 196 | 222 | 250 | 208 | 234 | 265 | |

| | |
|---|---|
| F (%) (90% CI)^{#} | 92% (0.86, 0.97) |

^{†}C_{coi} (ng/mL) for 10 mg. IV.
^{‡}Median.
^{§}Harmonic Mean.
^{ϑ}CL (mL/min) for 10 mg IV.
^{#}Bioavailability relative to 10 mg I.V. reference based on pooled dose-adjusted (to 10 mg) oral AUC_{0-∞} values.
^{††} Dose proportionality assessment of oral treatments only.

FIG. 1 shows the arithmetic mean plasma concentration-time profiles of gaboxadol following single oral doses (2.5, 5, 10, 15, and 20 mg) with horizontal lines Δ indicating the change between 6 and 12 hours. The bioavailability of gaboxadol is approximately 92%. Plasma AUC_{0-∞} and Cₘₐₓ of gaboxadol show dose proportional increases and appear to be linear over the entire dose range examined, from of 2.5 to 20 mg. The time to peak plasma concentrations (Tₘₐₓ 30-60 min) and the half-life (t½ of 1.5 h) for gaboxadol appear to be independent of dose across the gaboxadol dose range of 2.5 to 20 mg. The excretion of gaboxadol is mainly via urine, where 96.5% of the dose is recovered; 75% is recovered within 4 hours after administration.

### Example 2

Assessment of the Effectiveness of Gaboxadol in Patients with Narcolepsy

This prospective study will be used to evaluate the dose-dependent ability of gaboxadol to relieve symptoms of narcolepsy in adults between 18 and 65 years of age with a diagnosis of narcolepsy with or without cataplexy. Specifically, once-daily gaboxadol administered orally at 5 mg, 10 mg and 20-mg doses will be compared to placebo and will assess the magnitude and rate of response to gaboxadol as measured by the Epworth Sleepiness Scale (ESS), Maintenance of Wakefulness Test (MWT) and polysomnograms.

The trial will be conducted over 4 weeks, with double-blind treatment based on random assignment to gaboxadol or placebo. Approximately 60 adult patients with narcolepsy with or without cataplexy will be recruited for the study. Patients who meet study criteria will need to stop taking their current narcolepsy and/or other medication for at least 7 days before starting treatment. They will be randomly assigned to one of 4 treatment arms; each patient has an equal chance of receiving placebo, 5 mg, 10 mg and 20-mg gaboxadol.

After randomization the participants will be placed into 4 separate treatment groups (A-D) and a placebo group (E). Treatment group A receives 5 mg gaboxadol in the evening. Treatment group B receives 10 mg gaboxadol in the evening. Treatment group C receives 10 mg gaboxadol in the evening and 10 mg gaboxadol in the morning. Treatment group D receives 20 mg gaboxadol in the evening. Treatment group E receives placebo in the evening.

Throughout the 4 week study, medical history, physical examination, vision tests, blood pressure, heart rate, temperature, and ECGs will be checked periodically. Blood samples will be taken for standard safety laboratory tests as well as for the measurement of the drug blood level. The Maintenance of Wakefulness Test will be done to assess the patient's ability to resist falling asleep while reclining in a dark, quiet room; this will involve completing 7 sessions before dosing and 10 sessions after dosing. Efficacy will be evaluated by determining the change from baseline in sleep latency on the MWT, with sleep latency defined as the time from lights out until the first of three consecutive epochs of Stage N1 sleep or 1 epoch of N2, N3, or REM. Additional efficacy outcomes will include the ESS, which will be completed by the patient at baseline and at the weekly study visits (weeks 1, 2, 3, and 4), and the Clinical Global Impression of Change (CGI-C), which will be completed by the investigator at each weekly study visit. The CGI-C consists of a seven-point scale ranging from “1=very much improved” to “7=very much worse.” Patients will have two polysomnograms a week. A polysomnogram is a test of brain, muscle and eye activity during sleep, obtained by recording brain waves and other activities such as muscle and eye movement. Polysomnograms will be obtained overnight, once before and once after dosing. Patients will also complete brief questionnaires about their sleepiness and the status of their narcolepsy throughout the study.

Those skilled in the art will recognize, or be able to ascertain using no more than routine experimentation, many equivalents to the specific embodiments described herein. Such equivalents are intended to be encompassed by the claims.

What is claimed is:

1. A method of treating narcolepsy comprising administering to a patient in need thereof a pharmaceutical composition comprising gaboxadol or a pharmaceutically acceptable salt thereof.

2. The method of claim 1, wherein the patient is administered 0.1 mg to 30 mg gaboxadol or a pharmaceutically acceptable salt thereof.

3. The method of claim 1, wherein gaboxadol or a pharmaceutically acceptable salt thereof is administered to the patient in a daily dosage ranging from 1 mg to 30 mg.

4. The method of claim 1, wherein the pharmaceutical composition comprises 5 mg to 20 mg gaboxadol.

5. The method of claim 1, wherein gaboxadol or a pharmaceutically acceptable salt thereof is administered in a dose ranging from 0.1 mg/kg to 1 mg/kg.

6. The method of claim 1, wherein the composition is administered once, twice, three times daily, or every other day.

7. The method of claim 1, further comprising administering a compound selected from the group consisting of a CNS stimulant, a eugeroic agent, an antidepressant and a GABA_{B} receptor agonist.

8. The method of claim 7, wherein the CNS stimulant is selected from the group consisting of amphetamine, dextroamphetamine, methamphetamine, methylphenidate, phentermine, diethylpropion, phendimetrazine, lisdexamfetamine, benzphetamine, atomoxetine, caffeine and ephedrine.

9. The method of claim 7, wherein the eugeroic agent is selected from the group consisting of modafinil and armodafinil.

10. The method of claim 7, wherein the antidepressant is selected from the group consisting of dopamine active anti-depressant agents, dopamine active augmenting agents, serotonin-a norepinephrine reuptake inhibitors, norepinephrine reuptake inhibitors, monoamine oxidase inhibitors, tricyclic antidepressants, tetracyclic antidepressants and selective serotonin re-uptake inhibitors.

11. The method of claim 7, wherein the GABA_{B} receptor agonist is selected from the group consisting of sodium oxybate, baclofen and phenibut.

12. A method of treating narcolepsy comprising administering gaboxadol or a pharmaceutically acceptable salt thereof to a patient diagnosed with narcolepsy in an amount effective to reduce one or more symptoms of narcolepsy.

13. The method of claim 12, wherein the symptoms of narcolepsy are selected from the group consisting of excessive daytime sleepiness, abnormal REM sleep, cataplexy, sleep paralysis, hallucinations, automatic behaviors and night-time wakefulness.

14. The method of claim 12, wherein gaboxadol or a pharmaceutically acceptable salt thereof is administered in an amount ranging from 5 mg to 30 mg.

15. The method of claim 12, further comprising administering a compound selected from the group consisting of a CNS stimulant, a eugeroic agent, an antidepressant and a GABA_{B} receptor agonist.

16. A method of treating narcolepsy comprising administering to a patient in need thereof gaboxadol or a pharmaceutically acceptable salt thereof, wherein the method provides improvement in next day functioning of the patient.

17. The method of claim 16, wherein the patient exhibits a reduced amount of excessive daytime sleepiness.

18. The method of claim 16, wherein gaboxadol or a pharmaceutically acceptable salt thereof is administered in an amount ranging from 5 mg to 30 mg.

19. The method of claim 16, further comprising administering a compound selected from the group consisting of a CNS stimulant, a eugeroic agent, an antidepressant and a GABA_{B} receptor agonist.
